(19) United States
(12) Patent Application Publication
(10) Pub. No.: US 2023/0053201 A1
(43) Pub. Date: Feb. 16, 2023

(54) SYSTEMS AND METHODS FOR GENERATING NITRIC OXIDE USING MICROWAVE ENERGY

(71) Applicant: Third Pole, Inc., Waltham, MA (US)

(72) Inventors: Christopher Miles, Acton, MA (US); Ali Shajii, Weston, MA (US); Donald K. Smith, Newton, MA (US); Benjamin J. Apollonio, Lunenburg, MA (US); Gregory W. Hall, Belmont, MA (US)

(73) Assignee: Third Pole, Inc., Waltham, MA (US)

(21) Appl. No.: 17/819,582

(22) Filed: Aug. 12, 2022

Related U.S. Application Data
(60) Provisional application No. 63/232,993, filed on Aug. 13, 2021.

(51) Int. Cl. C01B 21/32; B01J 19/12

(52) U.S. Cl. CPC C01B 21/32; B01J 19/126

(57) ABSTRACT

System and methods are provided for generating nitric oxide (NO). In some embodiments, systems comprise a microwave generator configured to produce microwave energy of varying pulse duration, pulse frequency, and power level and a microwave cavity configured to utilize microwave energy to generate a plasma ball within a flow of reactant gas containing nitrogen and oxygen flowing through the microwave cavity to produce a product gas containing NO. At least one stub can be positioned in the microwave cavity and is configured to focus the microwave energy at a location at which the plasma ball is formed. A controller in electrical communication with the microwave generator can be configured to control the microwave generator to initiate and maintain the plasma ball so the plasma ball is suspended in the flow of reactant gas and does not contact a surface of the at least one stub and the microwave cavity.

### RELATED APPLICATIONS

This application claims the benefit of and priority to U.S. Provisional Application No. 63/232,993 filed Aug. 13, 2021, which is incorporated herein by reference in its entirety.

### FIELD

The present disclosure relates to the generation of nitric oxide, and more particularly to systems and methods for generating nitric oxide using microwave energy.

### BACKGROUND

Nitric oxide (NO) has been found to be useful in a number of ways for treatment of disease, particularly cardiac and respiratory ailments. Previous systems for producing NO and delivering the NO gas to a patient have a number of disadvantages. For example, tank-based systems required large tanks of NO gas at a high concentration and are required to purge with NO when treatment is resumed. Synthesizing NO from NO₂ or N₂O₄ requires the handling of toxic chemicals. Prior electric generation systems involve generating plasma in the main flow of air to be delivered to patients, or pumped through a delivery tube.

Microwave energy can also be used for NO generation, but require the use of high-voltage transformer circuits with electrodes for generating NO using electrical discharge. One major disadvantage to high voltage/electrode systems is that the electrodes wear over time due to oxidation and erosion, and eventually require replacement. In medical instrument applications, this requires the electrodes to be replaced after the instrument is placed in service for period of time. Another disadvantage is that replaceable electrodes may be constructed at least in part from precious metals such as iridium or platinum and may be costly.

### SUMMARY

The present disclosure relates to systems, methods, and devices for generation of nitric oxide (NO). In some embodiments, the system comprises a microwave generator configured to produce microwave energy of varying pulse duration, pulse frequency, and power level and a microwave cavity configured to utilize the microwave energy to generate a plasma ball within a flow of reactant gas containing nitrogen and oxygen flowing through the microwave cavity to produce a product gas containing NO. At least one stub can be positioned in the microwave cavity and configured to focus the microwave energy at a location at which the plasma ball is formed. A controller in electrical communication with the microwave generator can be configured to control the microwave generator to initiate and maintain the plasma ball such that the plasma ball is suspended in the flow of reactant gas and does not contact a surface of the at least one stub and the microwave cavity.

In some embodiments, the controller is configured to control a concentration of NO in the product gas using one or more control parameters to adjust at least one of the pulse duration, the pulse frequency, and the power level of the microwave energy and a reactant gas flow rate, the control parameters being related to at least one of the reactant gas, the product gas, an inspiratory gas into which at least a portion of the product gas flows, a prescribed amount of NO, and a patient receiving at least the portion of the product gas.

In some embodiments, the system can also include a reaction chamber, or plasma chamber, positioned within the microwave cavity such that the plasma ball is positioned within the reaction chamber. In some embodiments, the reaction chamber is configured to have an independent volume of gas therein. In some embodiments, the reaction chamber is configured to have an independent volume of gas therein that is less than a volume of gas within the microwave cavity. In some embodiments, the volume of gas in the reaction chamber allows for a decrease in transit time and NO2 formation due to the volume of gas in the reaction chamber being less than the volume of gas in the microwave cavity.

In some embodiments, the system can also include a vacuum chamber associated with the reaction chamber to initiate the plasma ball below atmospheric pressure. In some embodiments, the system can also include a valve upstream of the microwave cavity and a pump downstream of the microwave cavity, the valve and the pump working in combination to decrease a pressure in the microwave cavity. In some embodiments, the controller is configured to control one or more of the valve and the pump to control the pressure in the microwave cavity.

In some embodiments, the reactant gas includes NO to facilitate plasma formation. In some embodiments, the system can also include a cooling component configured to cool the reactant gas to increase NO production. In some embodiments, a temperature of the reactant gas is reduced up to 50° C. In some embodiments, the microwave generator includes a first antenna configured to initiate a plasma and a second antenna configured to sustain the plasma.

In some embodiments, a system for generation of nitric oxide is provided and comprises a microwave generator configured to produce microwave energy of varying pulse duration, pulse frequency, and power levels, and a microwave cavity configured to utilize the microwave energy to generate a plasma ball within a flow of reactant gas containing nitrogen and oxygen flowing through the microwave cavity to produce a product gas containing NO. A reaction chamber can be positioned within the microwave cavity and has a gas volume less than a gas volume of the microwave cavity. At least one stub can be positioned in the microwave cavity and is configured to focus the microwave energy at a location inside the reaction chamber such that a plasma ball is formed therein. A controller in electrical communication with the microwave generator can be configured to control the microwave generator to initiate and maintain the plasma ball in the reaction chamber.

In some embodiments, the plasma ball is suspended in the flow of reactant gas and does not contact a surface of the at least one stub and the microwave cavity.

In some embodiments, the controller is configured to control a concentration of NO in the product gas using one or more control parameters to adjust at least one of the pulse duration, the pulse frequency, and the power level of the microwave energy, a reactant gas flow rate, and microwave cavity pressure, the control parameters being related to at least one of the reactant gas, the product gas, an inspiratory gas into which at least a portion of the product gas flows, and a patient receiving at least the portion of the product gas.

A method of generating nitric oxide (NO) is provided, and comprises generating a plasma ball from a flow of reactant gas through a microwave cavity using microwave energy directed therein for producing a product gas containing nitric oxide from the flow of the reactant gas through the microwave cavity, focusing the microwave energy to a focal point in the microwave cavity using at least one stub positioned in the microwave cavity such that focal point is the location of the plasma ball, and controlling, using a controller, an amount of nitric oxide in the product gas using one or more parameters as input to a control algorithm used the controller to control the generation of the plasma ball. The plasma ball is suspended in the flow of reactant gas and does not contact a surface of the at least one stub and the microwave cavity.

In some embodiments, focusing the microwave energy further comprises focusing the microwave energy in a reaction chamber positioned within the microwave cavity such that the plasma ball is positioned within the reaction chamber.

In some embodiments, the controller is configured to control a concentration of NO in the product gas using one or more control parameters to adjust at least one of a pulse duration of the microwave energy, a power level of the microwave energy, a reactant gas flow rate, and reaction chamber pressure, the control parameters being related to at least one of the reactant gas, the product gas, an inspiratory gas into which at least a portion of the product gas flows, and a patient receiving at least the portion of the product gas.

In some embodiments, the method also includes cooling the reactant gas using a cooling component to increase NO production. In some embodiments, a temperature of the reactant gas is reduced up to 50° C.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 illustrates an exemplary embodiment of a NO generation system;

FIG. 2 depicts an embodiment of a NO generation and delivery system with a recirculation architecture;

FIG. 3 illustrates an exemplary embodiment of a reactor;

FIG. 4A and FIG. 4B depict a computational simulation of the field strength in a cavity for a particular antenna design;

FIG. 5 depicts an embodiment of a cylindrical microwave cavity with no flow guide;

FIG. 6 depicts an embodiment of a microwave cavity;

FIG. 7 depicts an exemplary embodiment of a microwave cavity 160 having an aluminum housing;

FIG. 8A and FIG. 8B illustrates exemplary graphs showing output from electromagnetic models of inside the microwave cavity;

FIG. 9 depicts an exemplary embodiment of a microwave NO generator with a reaction chamber within the microwave cavity;

FIG. 10 depicts exemplary components of RF drive circuitry that can be used in a process for generating the RF signal for generating a plasma ball;

FIG. 11 depicts an exemplary embodiment of a block diagram of a microwave plasma ball NO generation device;

FIG. 12A depicts an exemplary embodiment of a microwave NO production system;

FIG. 12B depicts an exemplary embodiment of a microwave cavity and a plasma chamber;

FIG. 13 is an exemplary photograph of a plasma ball during operation of a microwave NO generator;

FIG. 14 illustrates an example of the output of a computational fluid dynamics (CFD) model of a plasma chamber;

FIG. 15 illustrates an exemplary graph showing NO production values from a system designed to delivery up to 300 ppm.slpm NO;

FIG. 16 illustrates an exemplary graph showing NO production values from the same 300ppm.slpm system as FIG. 15;

FIG. 17 illustrates an exemplary graph showing power control variables;

FIG. 18 illustrates an exemplary graph showing the NO/NO2 ratio for an exemplary microwave NO generator;

FIG. 19A illustrates an embodiment of a microwave cavity that varies location of a nozzle;

FIG. 19B illustrates an embodiment of a microwave cavity that varies shape of a nozzle;

FIG. 19C illustrates an embodiment of a microwave cavity that modulates inlet flow;

FIG. 19D illustrates an embodiment of a microwave cavity that modulates mass flow rate of reactant gas;

FIG. 20 depicts an embodiment of a system where the product gas exiting a combination of a microwave cavity and a plasma chamber;

FIG. 21 depicts an embodiment of an NO generation and/or delivery system that includes first and second product gas pathways;

FIG. 22 depicts an exemplary embodiment of a system having a combination of a microwave cavity and a plasma chamber;

FIG. 23 illustrates an exemplary graph showing the performance of an embodiment of a microwave NO generation device;

FIG. 24 depicts an exemplary graph showing two approaches to providing a required range of NO production from a microwave plasma ball generator;

FIG. 25 depicts an exemplary graph showing the performance of a NO generation system for a spectrum of excitation frequencies;

FIG. 26 depicts an exemplary embodiment of a scrubbing portion of a NO generation device;

FIG. 27 illustrates a graph of the performance of an exemplary microwave plasma NO generator operating at 150 ml/min reactant gas flow and 800 Hz;

FIG. 28 depicts an exemplary embodiment of a microwave cavity with a window in the wall of the cavity;

FIG. 29 depicts an exemplary embodiment of a microwave cavity with an internal light sensor;

FIG. 30 depicts an exemplary flow profile of reactant gas as it passes through the path defined by the gas flow guides and inlet stub in a microwave cavity;

FIG. 31 is an exemplary photograph of the end of a 4 mm diameter aluminum inlet stub after multiple hours of NO generation;

FIG. 32A illustrates an exemplary microwave cavity that utilizes a two-piece flow guide;

FIG. 32B depicts an exemplary microwave cavity where reactant gas is introduced to the system through the inlet stub;

FIG. 33 illustrates a graph showing the results for three exemplary NO production levels;

FIG. 34 depicts an exemplary system that lowers pressure within the plasma chamber to facilitate plasma initiation;

FIG. 35 depicts an exemplary architecture with a combination microwave cavity and plasma chamber;

FIG. 36 depicts an embodiment of a NO generator with a flow restriction before the combination microwave cavity and plasma chamber;

FIG. 37 illustrates an embodiment of a microwave plasma ball generator with a pressurized scrubber architecture;

FIG. 38 depicts an embodiment of a system with a process controller and microwave generator directly connected to a microwave cavity;

FIG. 39 depicts an exemplary graph showing the performance of a representative NO generator operating at 12 W input power with separate microwave cavity and reaction chamber;

FIG. 40 depicts a graph showing exemplary NO/NO2 performance for an embodiment of the system for a variety of pulse frequencies and duty cycles; and

FIG. 41 depicts a graph showing the performance of an exemplary system with applied power on the X axis, NO production on the left vertical axis and NO/NO2 ratio on the right vertical axis.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments.

Specific details are given in the following description to provide a thorough understanding of the embodiments. However, it will be understood by one of ordinary skill in the art that the embodiments may be practiced without these specific details. For example, systems, processes, and other elements in the presently disclosed embodiments may be shown as components in block diagram form in order not to obscure the embodiments in unnecessary detail. In other instances, well-known processes, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed, but could have additional steps not discussed or included in a figure. Furthermore, not all operations in any particularly described process may occur in all embodiments. A process may correspond to a method, a function, a procedure, a subroutine, a subprogram, etc. When a process corresponds to a function, its termination corresponds to a return of the function to the calling function or the main function.

Subject matter will now be described more fully with reference to the accompanying drawings, which form a part hereof, and which show, by way of illustration, specific example aspects and embodiments of the present disclosure. Subject matter may, however, be embodied in a variety of different forms and, therefore, covered or claimed subject matter is intended to be construed as not being limited to any example embodiments set forth herein; example embodiments are provided merely to be illustrative. The following detailed description is, therefore, not intended to be taken in a limiting sense.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure relates to systems and methods of nitric oxide (NO) generation and/or delivery for use in various applications, for example, inside a hospital room, in an emergency room, in a doctor’s office, in a clinic, and outside a hospital setting as a portable or ambulatory device. An NO generation and/or delivery system can take many forms, including but not limited to a device configured to work with an existing medical device that utilizes a product gas, a stand-alone (ambulatory) device, a module that can be integrated with an existing medical device, one or more types of cartridges that can perform various functions of the NO system, and an electronic NO tank. The NO generation system uses a reactant gas containing nitrogen and oxygen, including but not limited to ambient air, to produce a product gas that is enriched with NO.

A system and method are provided for generating nitric oxide (NO) using microwave. In some embodiments, generation of nitrogen compounds by microwave energy can be utilized to generate a plasma ball within a flow of reactant gas. In some embodiments, a NO generation and delivery system can comprise a microwave generator, a microwave cavity, a plasma chamber (reaction chamber), and a scrubber. In some embodiments, the system can also include a reactant gas pre filter/scrubber. In some embodiments, microwave energy can be delivered to a resonant microwave cavity directly with an antenna or indirectly through a wave guide. In some embodiments, microwave energy can produce NO and has no parts in the NO generation system that wear over time, requiring replacement, and can be fabricated from low-cost materials.

In addition, the system described herein uses less power from an energy source to produce a given level of NO production. In battery powered instrument applications, less power enables the use of a lower capacity energy source, which is also directly related to instrument size, weight and cost. For example, this can be used for mobile or ambulatory instrument applications, where a patient may carry or transport a device during NO therapy. Lower power consumption also directly relates to longer run-time on a consumable or rechargeable battery source. In some embodiments, the NO/NO2 output ratio may be tuned to optimal values for a desirable input power range. In instrument applications that contain a disposable scrubber cartridge to remove NO2 from the plasma, the ability to optimize this value may be used extend the life of the disposable cartridge.

In some embodiments, a microwave cavity and a reaction chamber are concentric but have independent gas volumes. This decreases transit time for NO product gas to minimize oxidation and expedites the transit time to the quenching step. In some embodiments, the gas volumes are in fluid communication. In some embodiments, the gas composition and/or flow rate within the two volumes differs. In some embodiments, a continuous plasma ball is maintained with pulsed or continuous microwave energy. In some embodiments, an intermittent plasma ball formation is used (pulsed ball). In some embodiments, plasma initiation can include the use of low pressure (vacuum) and/or ambient pressure. In some embodiments, ultraviolet light is utilized to facilitate plasma formation due to ionization of the reactant gas.

An NO generation device can be used with or integrated into any device that can utilize NO, including but not limited to a ventilator, an anesthesia device, a defibrillator, a ventricular assist device (VAD), a Continuous Positive Airway Pressure (CPAP) machine, a Bilevel Positive Airway Pressure (BiPAP) machine, a non-invasive positive pressure ventilator (NIPPV), a nasal cannula application, a nebulizer, an extracorporeal membrane oxygenation (ECMO), a bypass system, an automated CPR system, an oxygen delivery system, an oxygen concentrator, an oxygen generation system, and an automated external defibrillator AED, MRI, and a patient monitor. In addition, the destination for nitric oxide produced can be any type of delivery device associated with any medical device, including but not limited to a nasal cannula, a manual ventilation device, a face mask, inhaler, or any other delivery circuit. The NO generation capabilities can be integrated into any of these devices, or the devices can be used with an NO generation device as described herein.

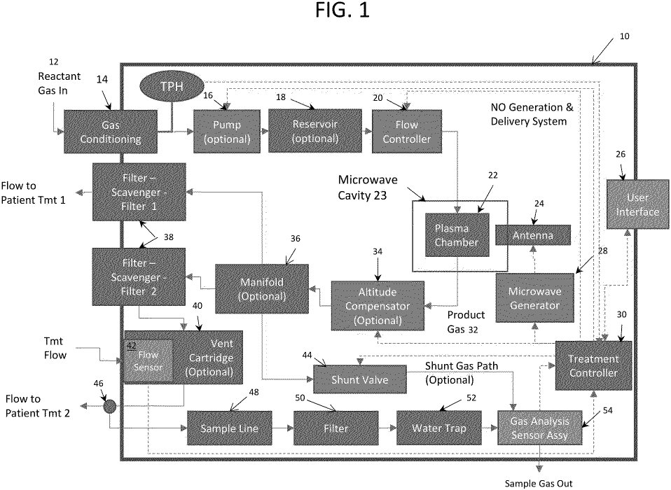

FIG. 1 illustrates an exemplary embodiment of a NO generation system 10 that includes components for reactant gas intake 12 and delivery to a plasma chamber 22. Reactant gas enters the system through a gas conditioning cartridge that includes one or more of a chemical scrubber (e.g., VOCs, NO2, ammonia), particulate filter, and one or more humidity adjustment mechanisms. A pressure, temperature, and/or humidity (PRT) sensors can characterize the physical properties of the reactant gas. This information is transferred to the treatment controller for input into the NO generator’s calculation of microwave activity, plasma chamber pressure and reactant gas flow rate to achieve a target level of NO production. The system is configured to produce, with the use of a microwave generation circuit 28, microwave cavity 23, and one or more microwave source antennas 24, a product gas 32 containing a desired amount of NO from the reactant gas. The system includes a treatment controller 30 in electrical communication with the microwave generator 28 that is configured to control the concentration of NO in the product gas 32 using one or more control parameters relating to conditions within the system and/or conditions relating to a separate device for delivering the product gas to a patient and/or conditions relating to the patient receiving the product gas.

The controller 30 is also in communication with a user interface 26 that allows a user to interact with the system, enter a target NO dose level, view information about the system and NO production, and/or control parameters related to NO production.

In some embodiments, the NO system pneumatic path includes a pump pushing air through a manifold 36. In other embodiments, pressurized reactant gas is provided to the inlet of the NO generator from an external source (e.g., wall air, pressurized gas cylinder, etc.). The manifold is configured with one or more valves: three-way valves, binary valves, check valves, and/or proportional orifices. The treatment controller 30 controls pump power, gas flow rate, the power in the plasma, and/or the direction of the gas flow post-electrical discharge. By configuring valves, the treatment controller can direct gas to the manual respiration pathway, the ventilator pathway or the gas sensor chamber for direct measurement of NO, NO₂ and O₂ levels in the product gas. In some embodiments, respiratory gas (i.e., the treatment flow) can be directed through a ventilator cartridge that measures the mass flow of the respiratory gas and can merge the respiratory gas with NO product gas.

The output from the NO generation system in the form of the product gas 32 enriched with the NO produced in the plasma chamber 22 can either be directed to a respiratory or other device for NO delivery to a patient or can be directed to a plurality of components provided for self-test or calibration of the NO generation system. In some embodiments, the system collects gases to sample in two ways: 1) gases are collected from a patient inspiratory circuit near the patient and pass through a sample line 48, a filter 50, and a water trap 52, or 2) gases are shunted directly from the pneumatic circuit as they exit the plasma chamber 22. In some embodiments, product gases are shunted with a shunt valve 44 to the gas sensors after being scrubbed but before dilution into a patient airstream. In some embodiments, product gases are collected from an inspiratory air stream near the device and/or within the device post-dilution. Within the gas analysis portion of the device, the product gas passes through one or more sensors to measure one or more of temperature, humidity, concentrations, pressure, and flow rate of various gasses therein.

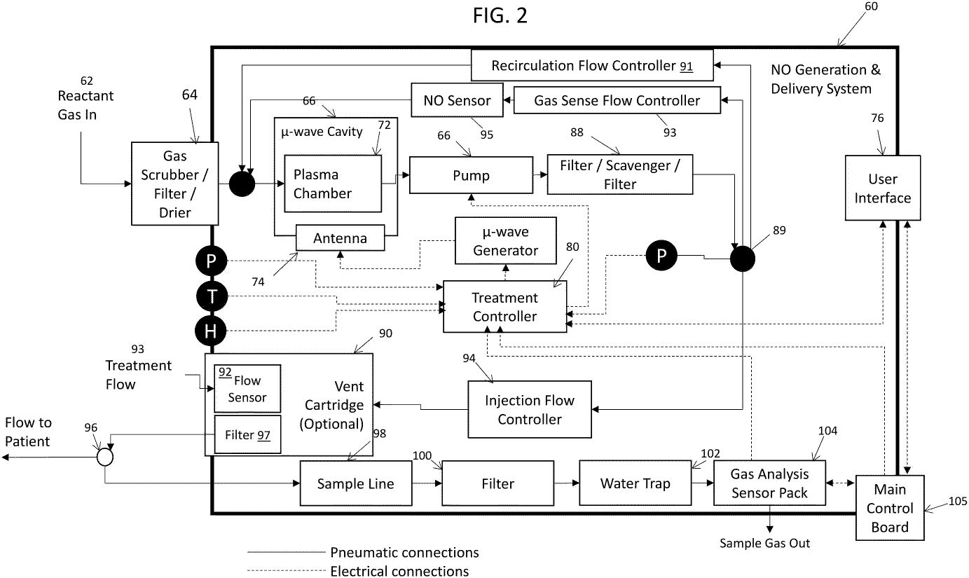

FIG. 2 depicts an embodiment of a NO generation and delivery system 60 with a recirculation architecture. Reactant gas 62 enters the system through a gas conditioner that includes one or more of a chemical scrubber, a particulate filter, and a humidifier/dehumidifier 64. Reactant gas passes through a junction where the recirculated gas enters the pathway and enters a plasma chamber. The plasma chamber is located within a microwave cavity that forms a plasma ball within the reactant gas flow path. A pump 66 is used to pull gas through the plasma chamber. In some embodiments, the pump operates at a constant flow rate (e.g., 3 slpm). Gas enters the recirculation loop to make up for gas injected into the patient inspiratory pathway. Gas exiting the pump passes through a NO₂ scrubber to eliminate NO₂ formed by the NO generation process and NO₂ formed from oxidation of NO as it travels around the recirculation loop. The NO₂ scrubber includes one or more filters before and/or after it to prevent migration of scrubber material and capture particles released from the scrubber and potentially other parts of the system (e.g., sputtered particles from stubs). After exiting the scrubber/filter stage, the gas enters a node that is maintained at constant pressure, as measured by pressure sensor 81. There are two or more flow paths exiting the constant pressure node. One flow path leads to a flow controller that controls product gas injection into the patient inspiratory stream. The node is maintained at constant pressure to facilitate and improve the accuracy of the injection flow controller. A second flow path and flow controller can be utilized to maintain a target pressure at the node and return product gas to the beginning of the recirculation loop (pre-plasma chamber). A third and optional flow path includes a flow controller that regulates the product gas flow through one or more gas sensors used to monitor the NO and/or NO₂ levels within the product gas. A microwave antenna 74 within the resonant microwave cavity 72 is energized by a microwave generator 78 that produces microwaves of varying pulse duration and power level based on desired treatment conditions received from a treatment controller 80. A user interface 76 receives desired treatment conditions (dose, treatment mode, etc.) from the user and communicates them to the main control board 105. The user interface is also used to communicate device status to the user (e.g., alarm conditions, disposable component status, battery charge status, alarm silence, and other types of information). The main control board 105 relays to the treatment controller 80 a target dose and monitors measured NO concentrations from the gas analysis sensor pack 104. The main control board 105 monitors the system for error conditions and can generate alarms, as required. Reactant gas 62 is converted into product gas 82 when it passes through the plasma chamber 72 and is at least partially converted into nitric oxide and nitrogen dioxide. Ambient gas measurements made by pressure, temperature and/or humidity sensors on the left edge of the image are used as inputs to the treatment controller for plasma compensation to improve dose accuracy. In some embodiments, conditioning of the reactant gas is actively controlled by the controller (e.g., degree of dehumidification). In some embodiments, the treatment controller increases the duty cycle and/or frequency of the microwave pulses as humidity increases from 10% to 50%, for example, to maintain a constant production level of NO. In some embodiments, the treatment controller decreases the microwave pulse duty cycle and/or frequency as pressure increases in the plasma chamber to maintain a constant production level of NO. Product gas exits the plasma chamber 72 and passes through a pump 66, a NO2 scrubber 88 and a particle filter 88. In some embodiments, the product gas flow rate through the pump is constant to facilitate greater NO production control. The flow rate through the plasma chamber may vary from 100 ml/min to 5 lpm, depending on the application and quantity of NO to be generated. In some embodiments, the chamber geometry is designed for optimal NO production at a particular reactant gas flow rate and the system varies reactant gas flow rate as a means to modulate NO production.

Product gas exiting the particle filter enters a node 89 where it can travel in three different directions. Product gas flows through an injection flow controller 94 prior to introduction to a treatment gas flow. Excess product gas beyond what is required for dosing the treatment gas is routed through a recirculation flow controller 91 that is controlled to maintain a constant product gas pressure at the node 89. In some embodiments, a third gas flow path routes product gas through a gas sense flow controller 93 to provide a specific flow rate of gas for an NO sensor 89. Measurements from a NO sensor 95 are utilized by the treatment controller to one or more of maintain a target concentration of NO within the product gas and measure the amount of NO lost to the system (i.e., tubing walls, scrubber, etc.).

After exiting the injection flow controller 94, product gas enters a vent cartridge 90. In some embodiments, the vent cartridge 90 includes one or more mass flow sensors 92 that measure the treatment gas flow 93. The treatment gas flow measurements from the flow sensor 92 serve as an input into the product gas flow controller 94 via the treatment controller 80. After product gas 82 is introduced to the treatment flow, it passes through an optional filter 97 and inspiratory tubing. Near the patient, a fitting 96 is used to pull a fraction of inspired gas from the inspiratory flow, through a sample line 98, filter 100, water trap 102 and Nafion tubing (not shown) to prepare the gas sample and convey it to gas sensors 104. Gas sensors may measure one or more of nitrogen, oxygen, nitric oxide, nitrogen dioxide, helium, and carbon dioxide. Sample gas exits the gas analysis sensor pack 104 to ambient air. In some embodiments, the sample gas is scrubbed prior to release to the environment (not shown). In some embodiments, the system 60 can optionally direct gas through a shunt valve 94 and shunt gas path 95 directly to the gas sensor pack and out of the system. In some embodiments involving the shunt valve 94, the manifold 86 includes a valve (not shown) to block flow to the filter-scavenger-filter when the shunt valve 94 is open.

### Microwave Energy

Microwave energy is electromagnetic energy with a wavelength between 1 mm and 1 m or frequency of 300 GHz to 300 Mhz, respectively. Microwave energy is generated by a high frequency power supply and transmitted by an antenna. The energy can be introduced directly into a microwave cavity or directed through a waveguide from a remote generation location to the microwave cavity. In some embodiments, the microwaves operate within a sealed chamber while in other embodiments, the microwaves operate within a cavity that is shaped to focus microwave energy but permits the exchange of gas into and out of the cavity.

In some embodiments, a microwave NO generator will sweep through multiple operating frequencies to identify a resonant frequency of the cavity. In some embodiments, a microwave NO generator operates at a frequency at or near a resonant frequency. Resonance, especially with a high Q value, provides voltage gain that can be used to obtain high electric field strengths in the plasma ball region from a small input voltage. Higher RF signal amplitudes allow creation of the necessary electric field strengths with a lower Q in the resonant circuit because less resonant gain is needed to achieve the same voltage. Resonant circuits with a low Q have broader peaks and are therefore less sensitive to small variations in excitation frequency or component values, such as might occur due to changes in operating temperature.

Microwave frequency can influence cavity design. In some embodiments, the cavity design is cylindrical or spherical with dimensions that are a function of the microwave energy wavelength (e.g., ¼ wavelength) to produce predictable regions of concentrated microwave energy. With sufficient power applied, concentrated microwave energy can produce a plasma which can separate molecules into their monatomic and ionic forms. As an example, when air is present in the microwave cavity and a plasma is formed, diatomic nitrogen and oxygen are separated into individual nitrogen and oxygen atoms and ions that can recombine into new molecules.

Microwave energy is typically at frequencies from 300 MHz to 300 GHz, however the other frequencies outside this range can be used.

### Plasma Ball NO Generation

Focused, resonant microwave energy can generate sufficient energy within the reactant gas to form a plasma without the plasma directly contacting a solid surface. This enables a system to operate at lower component temperatures, minimizing the potential for long term/thermal degradation of the chamber internal components with reduced and potentially zero wear, greatly increasing service life over conventional electrical discharge plasma generators. Lower component temperatures can also result in the reduction of thermally-induced stresses and surface oxidation of NO generator internal components in and around the plasma. This approach also virtually eliminates the potential for sputtered metallic particles being introduced to the product gas, as can happen with electrical discharges between electrodes.

In some embodiments, the reaction chamber is designed so that the highest electric field occurs between the tip of the inlet stub and an outlet stub. In some embodiments, the outlet stub additionally serves as a gas outlet nozzle. This is where the resonant microwave energy is most dense and where plasma formation occurs. The plasma ball does not touch the inlet stub because the plasma ball exists in a region where power input from the electric field is high enough to overcome the losses by diffusion and to some extent radiation and convection. Diffusion losses to nearby surfaces such as the stubs are higher than the local electric field can support, limiting the extent of the plasma.

In other words, the plasma is sustained in a region where the power balance and particle balance have steady state solutions. Hence, the losses of energy (e.g., thermal conduction) and losses of ions and radicals to the inlet stub (e.g., recombination of ions or radicals at the surface of the stub) are balanced by the input power from the microwave field and the resulting ionization rate and dissociation rate in the core of the plasma. Near the inlet stub (i.e., material boundary) the losses are very high and even though the microwave field may be strong there, the losses will dominate and prevent sustaining of the plasma in the region near the stub surface. When the microwave fields are sufficient, the microwave energy absorption in the core of the plasma will generate ionization and radicals to balance the losses outward by diffusive processes, diffusion of heat and radicals and electrons.

Microwave plasma ball NO generation is a hot plasma approach to NO generation. A gap of air is maintained between the plasma ball and inlet stub. In some embodiments, reactant gas flows parallel to the axis of the inlet stub. When it reaches the end of the inlet stub, gas passes by the periphery of the plasma ball and is exposed to the plasma heat. In some embodiments, the plasma ball is maintained continuously with either pulsed or continuous microwave energy. In some embodiments, the plasma ball is formed and extinguished at a frequency and duration that delivers a target quantity of NO. Owing to the high gas temperatures, lack of corona (nanoseconds in duration at most), and existence of a continuous plasma ball during this process, ozone production is very low. In some embodiments, not shown, involve directing reactant gas at the plasma ball and/or tangent to the plasma ball. With each design, a balance is struck between the forces acting on the plasma ball to maintain the plasma and provide predictable, repeatable NO generation.

In some embodiments, the plasma ball is formed by the NO generator using continuous microwave energy (e.g., from initial start until fully formed) and then the NO generator switches to a pulsed energy mode to maintain the plasma ball, but to conserve input power to the NO generator. In some embodiments, the NO generator may use a first frequency (e.g., resonant, or other) to form the plasma ball and then use a second frequency to maintain the plasma ball. In some embodiments, the first frequency used may be the fundamental or near the fundamental resonant frequency and the second frequency may be a harmonic of the first frequency. In some embodiments, the first frequency and the second frequency may use different modulation frequencies.

In some embodiments, the physical size of the plasma ball has a direct correlation to the concentration of NO produced at a given flow rate. In other words, the diameter of the formed plasma ball not only corresponds to the microwave energy required to sustain the plasma ball, but also the concentration of NO produced by the generator at a constant flow rate (e.g PPM SLPM.

### Reactor Design

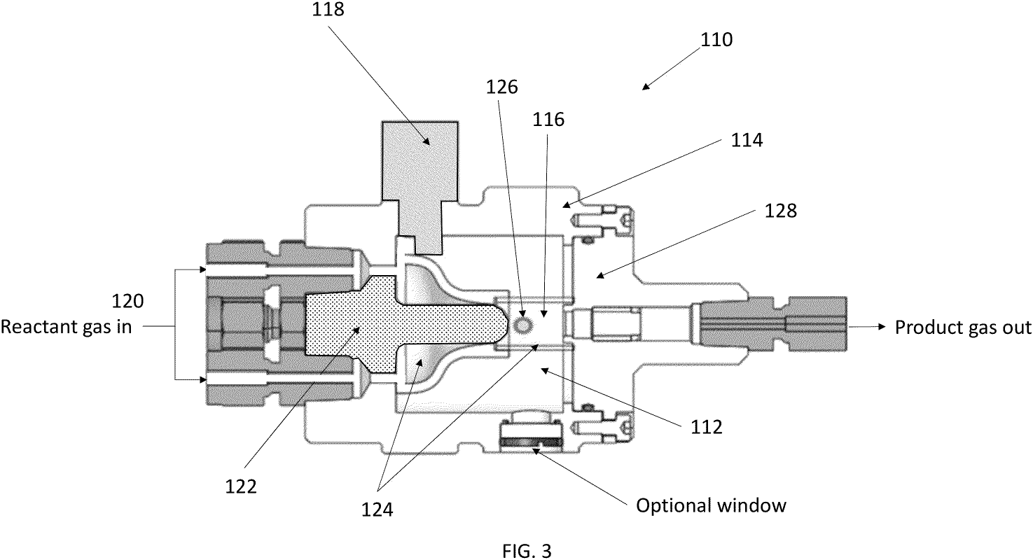

An exemplary reactor design is depicted in FIG. 3. The reactor 110 shown in FIG. 3 includes two concentric chambers. The outer chamber, a microwave cavity 112, is constructed of or plated with an electrically conductive material to contain microwave energy, and includes a microwave cavity housing 114. The inner chamber wall forms a plasma chamber 116. The inner chamber wall constructed of a microwave-permeable, gas-impermeable material (e.g., quartz, Teflon, ceramic) guides reactant gas through the plasma region. This approach provides necessary geometry for focusing microwave energy in a specific location for plasma formation within the reactant gas while also minimizing the volume that product gas travels through. This minimizes the transit time of product gas to minimize time for oxidation into NO₂ and expedites transit to the device outlet and ultimately to a patient.

Microwave energy enters the microwave cavity 112 from the microwave inlet 118. The microwave energy can either be made remotely and piped via a waveguide to the microwave cavity or be generated proximal to the cavity via antenna. In some embodiments, a microwave source antenna extends into the cavity. In some embodiments, a waveguide directs microwave energy into the cavity.

Reactant gas enters the reactor through two equivalent inlets 120. In some embodiments, the inlet is in the form of an annular space. The reactant gas flows around a central inlet stub 122, constrained by gas flow guides 124. Microwave energy is introduced through a side of the microwave cavity 112. The length of the cavity is selected to be approximately an odd multiple of one quarter of the microwave wavelength so that the highest electric field density occurs at the tip of the inlet stub. The gas outlet of the cavity forms a gas outlet stub to focus microwave energy at a point between the stubs. The temperature at the focal point exceeds temperatures high enough to ionize gas molecules, form plasma and disassociate N₂ and O₂ molecules into their monatomic form.

As reactant gas passes by the tip of the inlet stub, it flows past a plasma ball 126. A portion of the reactant gas is converted to nitric oxide and nitrogen dioxide. The gas flow guides 124 minimize the volume of the plasma chamber 116, which is less than the volume of the microwave cavity. In some embodiments, the gas flow guides are not used at all which can result in higher NO2 levels due to greater transit time for the product gas to exit the cavity. These effects may be negligible, depending on the reactant gas flow rate and cavity volume. In some embodiments, the reactant gas flow rate is in the range of 100 ml/min to 5 lpm, depending on the production levels required and system architecture.

As product gas exits the cavity, it travels through the outlet stub and past an optional heat exchanger 128 which quenches the product gas to protect downstream components from elevated product gas temperature. Product gas exits the right end of the figure where it can pass through the rest of the system and on to the patient.

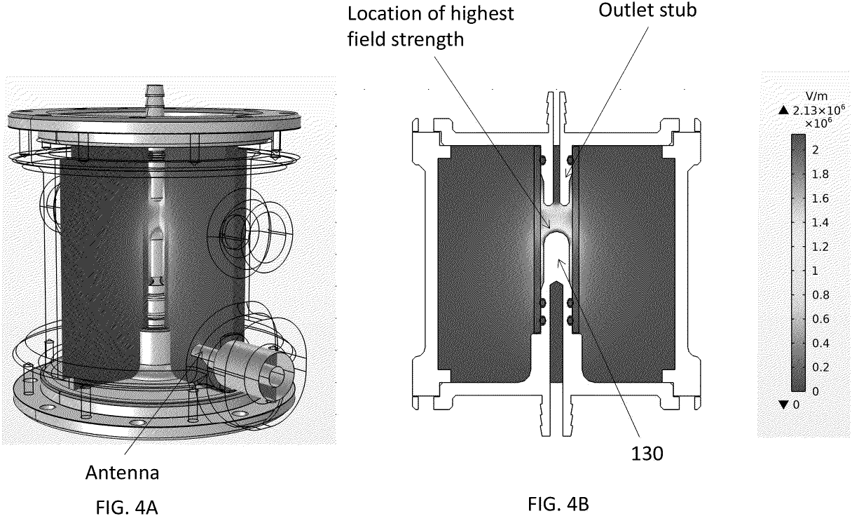

The cavity geometry of a microwave NO generator (i.e., body, top lid, bottom lids, quartz tube, and stubs) determines the resonator properties of the cavity. The internal dimensions of the cavity (radius and length), the stub geometries of the top and bottom lid, the quartz tube material properties (dielectric constant), material dimensions (wall thickness), and antenna design are the primary drivers of the resonator performance. In some embodiments, the cavity is designed to be resonant at 2.5 GHz, and to provide an adequate field strength between the top and bottom stubs to ensure sufficient voltage to initiate breakdown in air to ignite a plasma. An excitation frequency of 2.5 GHz is convenient due to its widespread use in wireless infrastructure and ease of availability of microwave signal generation and amplification components. Additionally, it produces reasonable cavity dimensions. FIG. 4A and FIG. 4B depict a computational simulation of the field strength in a cavity for a particular antenna design (in this case, an unmodified Amphenol RF 172270 connector). The field strength is highest along the top surface of an inlet stub 130. The main drivers are the cavity diameter and length as well as the bottom stub. The dimensions of these features will determine the natural resonant frequencies of the cavity for the various resonant modes that this type of cavity would exhibit. These frequencies may be affected by the addition of dielectric materials within the cavity walls, such as the quartz tube used to form the flow path.

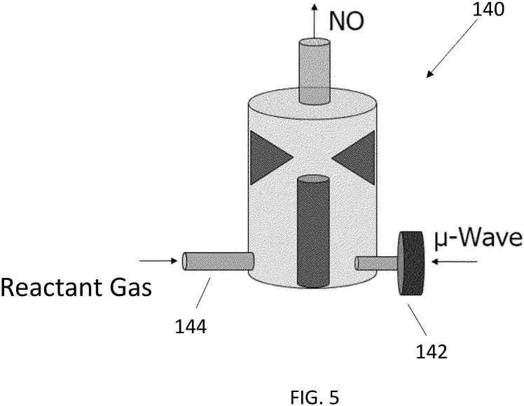

FIG. 5 depicts an embodiment of a cylindrical microwave cavity 140 with no flow guide. Microwave radiation enters the chamber at inlet 142. Reactant gas enters the chamber at inlet 144. In some embodiments, reactant gas enters the chamber parallel to a centered stub to establish laminar flow along the length of the center stub. In some embodiments, reactant gas flow is introduced tangent to the cylindrical chamber so that gas swirls around the inlet stub as it travels the length of the chamber. The absence of a flow guide can allow for simplicity and low mass.

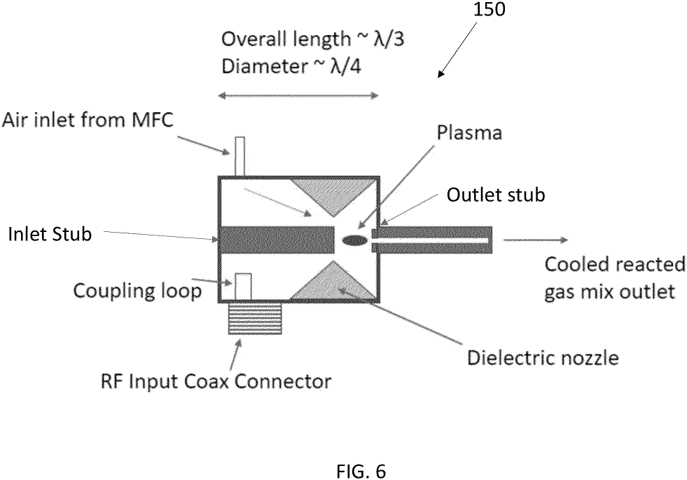

FIG. 6 depicts another embodiment of a microwave cavity 150. The length of the chamber and the diameter of the cavity are roughly ⅓ and ¼ the wavelength of the microwave radiation, respectively. For example, the internal diameter of the cavity can be 37.0 mm and the internal length can be 26.2 mm. As shown, a cooled product gas outlet can be used for quenching the product gas as it exits the microwave cavity, if so desired.

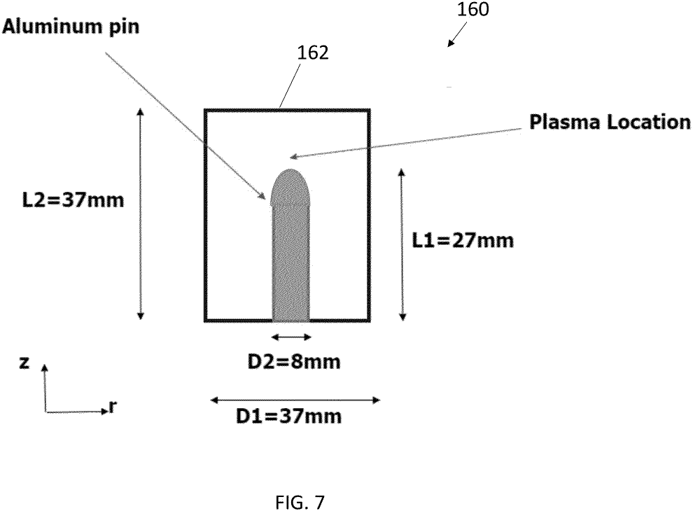

FIG. 7 depicts an exemplary embodiment of a microwave cavity 160 with additional dimensional details and having an aluminum housing 162. The resonant mode in the cavity is a modified TEM (transverse electromagnetic) coaxial line mode. It will be symmetric and transverse-magnetic, but have Ez and Er (radial and longitudinal electric fields). In some exemplary embodiments, the target resonant frequency is 2.45 GHz. It will be understood that that many possible cavity modes could be chosen to produce a desired microwave field.

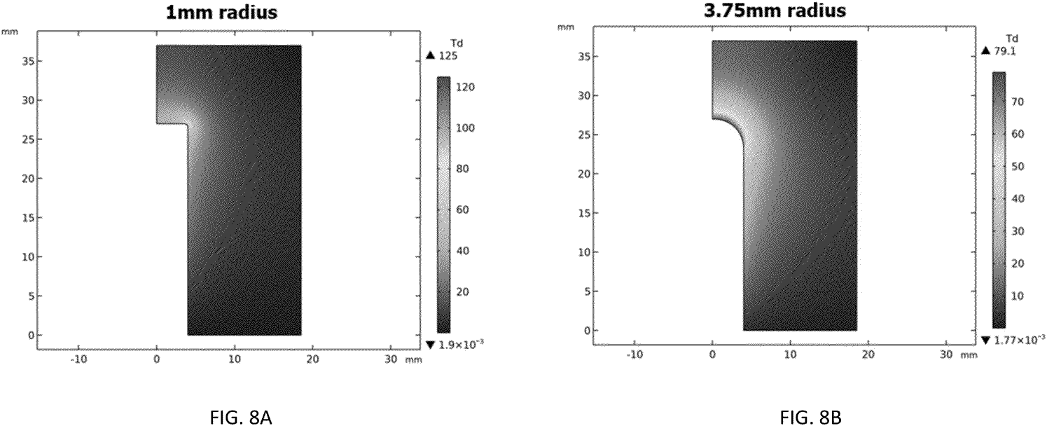

FIG. 8A and FIG. 8B illustrates exemplary graphs showing output from electromagnetic models of inside the microwave cavity. Peak electromagnetic field occurs at or near the edge of the end of the inlet stub. A smaller radius on the edge of the inlet stub results in a higher electric field.

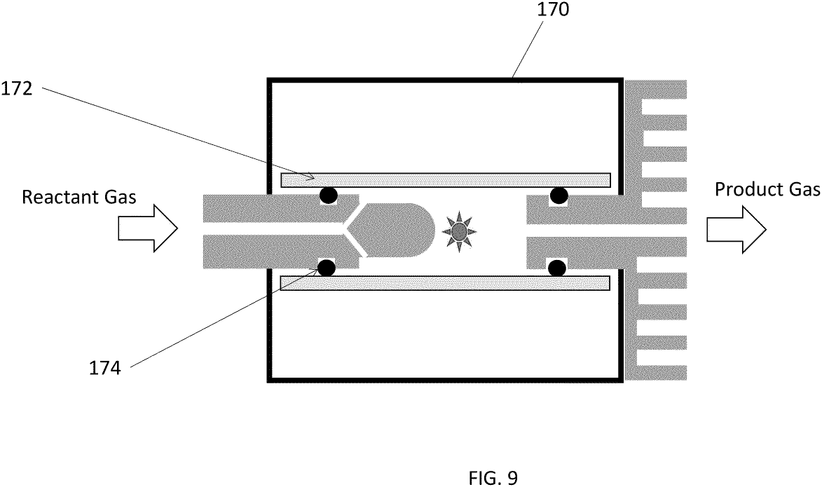

FIG. 9 depicts an exemplary embodiment of a microwave NO generator with a reaction chamber within the microwave cavity 170. The reaction chamber is comprised of a non-electrically-conductive flow guide or tube 172 sealed with O-rings 174 to the pin and outlet. One benefit of utilizing a reaction chamber within the microwave cavity is that the volume is low, thereby reducing transit time and potential for NO to oxidize. Reactant gas enters the central inlet stub through the center of the pin on the left side of the image. Reactant gas passes through the center of the stub and out through radial holes to an annular space defined between the stub and flow guide tube. The number of radial holes varies, for example, from 1 to eight or more. As the reactant gas passes through and over the central stub, the stub is convectively cooled. The chamber outlet serves as an outlet stub and is constructed of an electrically and thermally-conductive material (e.g., aluminum) with cooling fins to quench the product gas temperature. In some embodiments the aluminum is anodized or otherwise treated to reduce the potential for corrosion in the presence of NO₂.

### RF Signal Generation

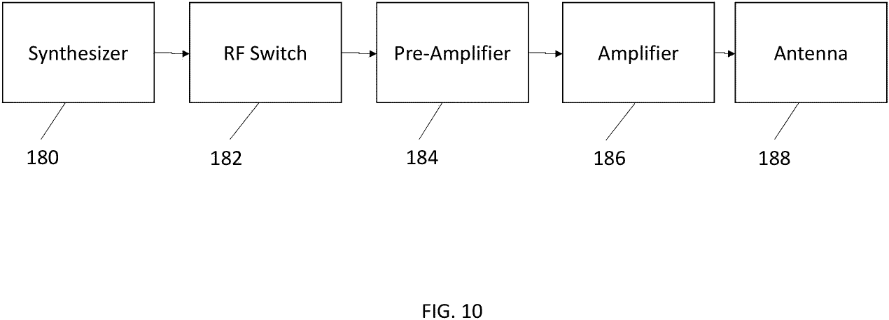

FIG. 10 depicts exemplary components of RF drive circuitry that can be used in a process for generating the RF signal that ultimately generates the plasma ball. A synthesizer 180 is utilized to generate the RF drive signal (RF switch 182) provided to a pre-amplifier 184. The synthesizer generates the RF carrier frequency. Using a synthesizer over other components, such as a fixed frequency oscillating chip, allows for modulation of the RF drive signal in response to one or more of changes in cavity resonance, NO production levels, reactant gas mixture, reactant gas humidity, and other factors that can alter NO production. In some embodiments, an integer-N synthesizer is utilized. The controller uses an RF switch to pulse modulate the RF output from the synthesizer at a designed frequency and duty cycle. In some embodiments, the modulation frequency ranges from 1 kHz to 30 kHz and the duty cycle ranges from 0.1% to 20%. The modulated RF signal passes through a preamplifier that generates a high amplitude RF signal. In some embodiments, the amplitude is approximately 27 V peak to peak. The preamplifier output then drives a power amplifier 186 that drives an antenna 188. The controller communicates with the synthesizer (e.g. SPI interface) to control the output frequency and duty cycle. In some embodiments, output is controlled within a frequency range of 2400 MHz to 2725 MHz. In some embodiments, the frequency is adjusted in steps of 0.5 MHz.

### System Design

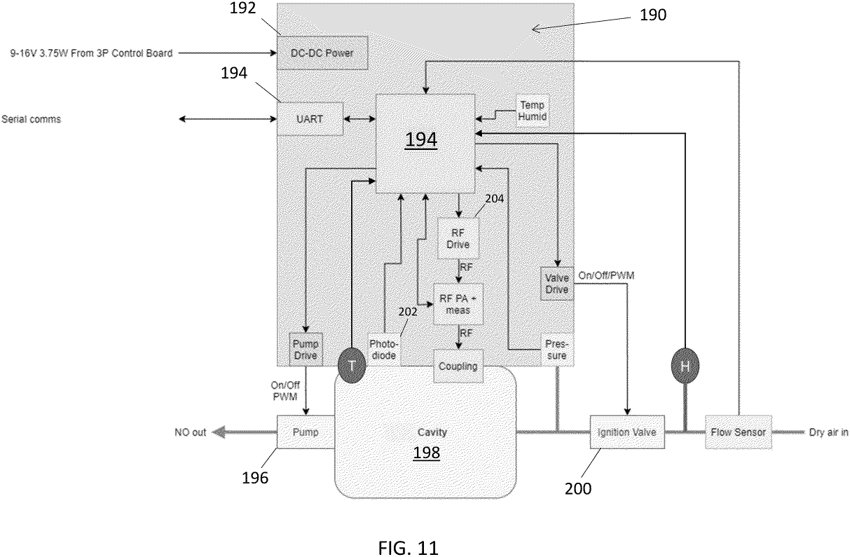

FIG. 11 depicts an exemplary embodiment of a block diagram of a microwave plasma ball NO generation device. A NO generation control board 190 receives DC power from an input 192. A microcontroller 194 manages operations of the device, utilizing a serial interface 194 to communicate with the overall device controller. The communication interface is utilized to receive information including but not limited to treatment settings, alarm conditions, device data (e.g., electrode age, scrubber type), environmental conditions, patient breath rate, and patient inspiratory flow rate. The communication interface is also utilized to send information to the overall device controller including but not limited to alarm conditions, NO production level, ambient conditions measurements, total run time (that can be used, for example, for determining service life), and other information.

The microcontroller controls a pump 196 that draws gas through the plasma cavity 198. In some embodiments, an ignition valve 200 is controlled to at least partially obstruct the inflow of product gas, thereby lowering the pressure within the plasma chamber to facilitate plasma ball initiation. In some embodiments (not shown), the system includes more than one reactant gas inlet valve and the system opens or closes one or more valves to vary the pressure within the plasma chamber. In some embodiments, the ignition valve is a proportional valve that can regulate the pressure and/or flow in the cavity. A reactant gas flow sensor is utilized to one or more of confirm reactant gas flow, detect an obstructed reactant gas flow path, detect a faulty pump, provide flow measurements to generate closed-loop control of the pump, and confirm proper operation of the ignition valve. For example, the reactant gas flow sensor can be used to confirm proper operation of the inlet valve. To pump the cavity down to vacuum, the inlet valve can be closed, which will cause the flow rate through the cavity to drop to 0. In combination with the pressure sensor reading, the data can be used to confirm that the inlet valve is working properly and the cavity pressure is being successfully reduced to facilitate fast, reliable ignition.

A photo diode 202 provides photons that destabilize gas within the region of plasma ball to facilitate initial gas excitation to a plasma state. The microcontroller 194 also controls an RF drive 204 that generates RF energy. The RF energy is introduced to the cavity through a coupling antenna. Various measurements are made of the RF energy by the microcontroller to manage the generation process. A pressure sensor is in fluid communication with the plasma chamber. Pressure measurements are utilized for one or more of closed-loop control of the ignition valve and/or pump and compensation of plasma activity based on actual plasma chamber pressure. In some embodiments, the pressure sensor is also utilized to detect one or more of an obstruction in the reactant gas flow path, a failed pump, a faulty ignition valve. In some embodiments, the microcontroller also utilizes plasma chamber temperature and reactant gas water content, as measured by a humidity sensor) in determining plasma parameters for accurate NO production. In some embodiments, plasma chamber temperature is measured by measuring the infrared output of the plasma chamber.

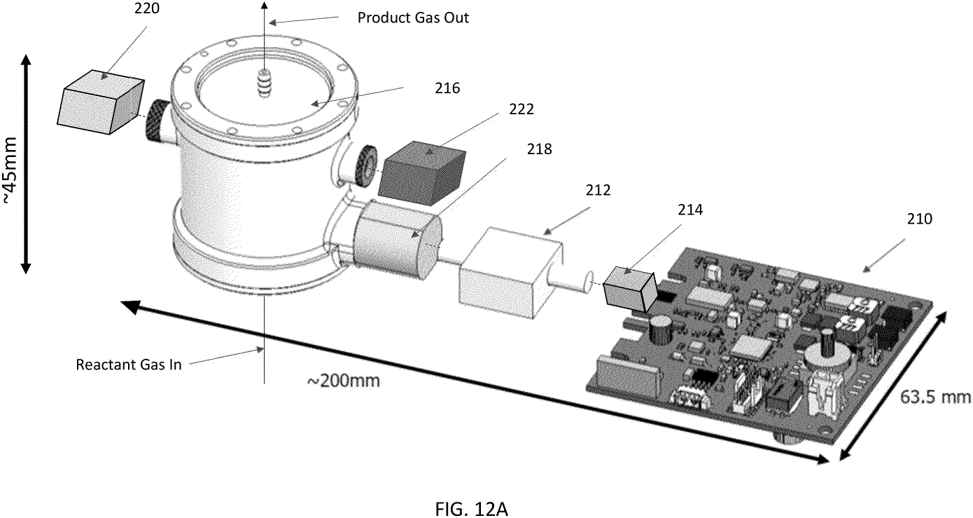

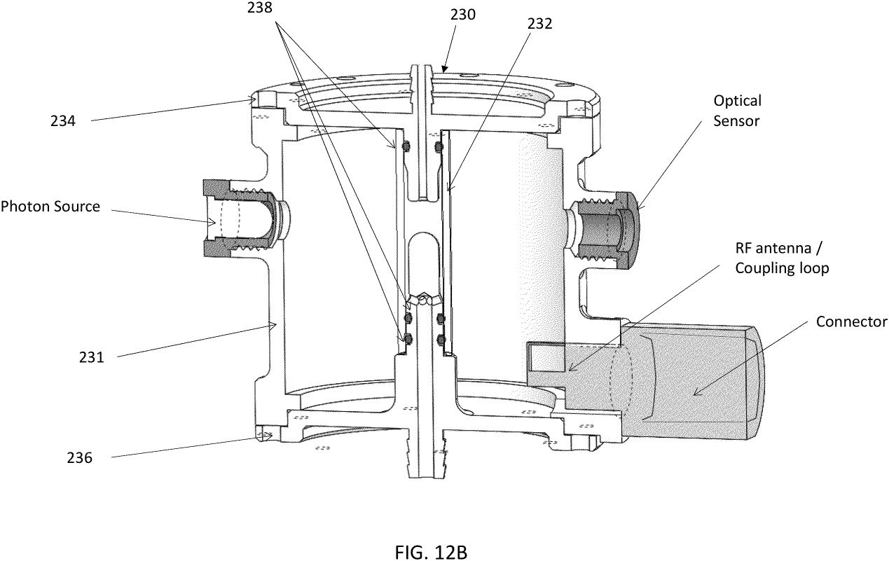

FIG. 12A depicts an exemplary embodiment of a microwave NO production system. On the right end is a circuit board for a controller 210. The controller 210 interfaces with a circulator component 212 via an RF coupling 214. Microwave energy entering the circulator 212 from the controller 210, which controls signal generation and amplification, is delivered to a microwave cavity 216. To protect the RF amplifier, microwave energy reflected from the cavity 216 to the circulator is eliminated by the circulator. The circulator component connects to an RF antenna/coupling loop 218 via a connector. The RF connector is inserted into the microwave cavity. A light source 220 (e.g., UV source) sends photons into the cavity 216 through a quartz window. On the opposite side of cavity, an optical sensor 222, such as a UV sensor, detects the of radiation in the UV and/or visible spectrum and is utilized to measure light output from the plasma for analysis by the controller. The timing and quantity of light output is indicative of proper function of the device and NO production. Product gas flow from the bottom to the top through pneumatic connections.

FIG. 12B depicts an exemplary embodiment of a microwave cavity 230 formed in a cavity body 231 and plasma chamber formed in by a plasma chamber wall 232. The top and bottom lids 234, 236, sealing O-rings 238, quartz tube dimensions and gas flow rate determine the airflow dynamics within the cavity. Each lid 234, 236 contains an air passage that is axially centered and allows air to flow through the stub features of the lids. The bottom lid contains cross-drilled holes (6 in this example) distributed radially around the stub (i.e., antenna) to evenly distribute the air. The gap between the interior wall of the quartz tube and the exterior surface of the stub on the bottom lid is tightly controlled to ensure sufficient flow velocity up along the bottom stub. In combination with the top lid and empty space between the stubs, the bottom stub geometry creates a recirculation zone around the plasma to help stabilize it and cool the outlet gases from the plasma. The top lid outlet passage also cools the outlet gases.

The sealing O-rings 238 provide the seal for the flow path, necessary to ensure the correct flow of gas through the plasma chamber and the ability to pull a vacuum on the plasma chamber. They also locate the quartz tube and ensure good concentricity between both lids and the quartz tube. This ensures a uniform gap between the quartz tube and the bottom lid, providing uniform flow characteristics around the outlet stub and therefore the plasma.

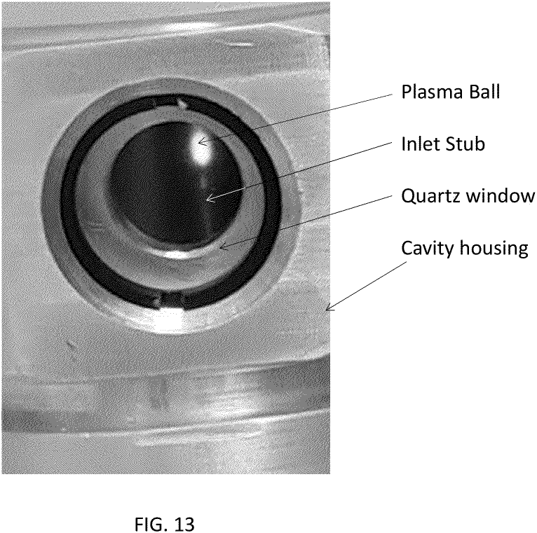

FIG. 13 is an exemplary photograph of a plasma ball during operation of a microwave NO generator. Reactant gas travels from the bottom to the top of the image. The bright plasma ball is evident at the top of the inlet stub

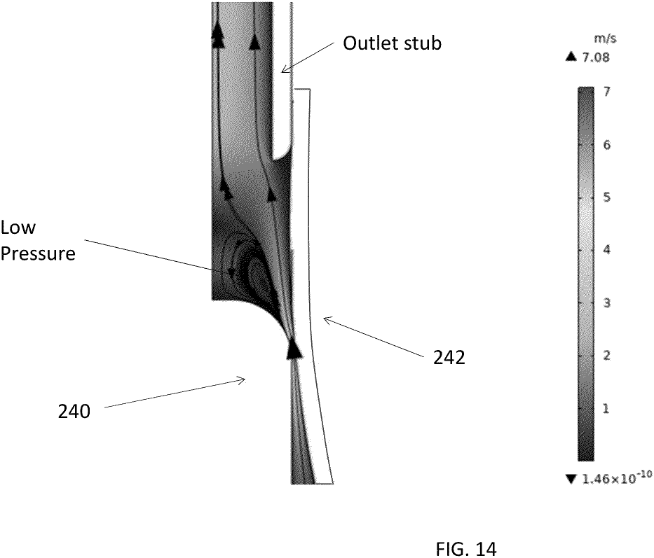

FIG. 14 presents an example of the output of a computational fluid dynamics (CFD) model of the plasma chamber. The image is a slice of the gas flow volume showing an inlet stub 240 and the inner wall of the plasma chamber wall 242 (e.g., quartz tube). The annular space between the quartz tube and inlet stub is smallest near the tip of the inlet stub, creating the highest reactant gas flow rates and directing gas in the longitudinal direction of the chamber. A low-pressure zone (i.e., eddy) is located at the tip of the inlet stub. The plasma resides in this low-pressure zone.

### NO Production Controls

Typical NO electric generators can produce NO at a range of levels. Factors that enable NO production modulation within the production range include power, duty cycle, frequency, gas flow rate, and gas/plasma interaction. These factors are controlled by a treatment controller. In some embodiments, the treatment controller is located within the device. In some embodiments, the treatment controller is located in an external or adjacent device.

### Plasma Power Control

Power: The power delivered to the reaction chamber can be varied to affect the plasma size and temperature, in turn affecting the amount of NO generated. In some embodiments, the controller measures the RF power using an optical sensor that measures the light output from the plasma. In some embodiments, the controller measures the input power to the RF power amplifier. In some embodiments, the controller measures the reflected signal strength from the coupling antenna to determine impedance match and power transfer.

RF Signal Amplitude: Plasma power can be modulated by changing the amplitude of the RF drive signal. There are various ways to modulate the RF amplitude. For example, synthesizer RF drive output strength (software adjustable via firmware) hardware attenuators in the signal path can be changed (discrete resistors being swapped out to form an attenuator with a different dB value), or plasma power can be modulated by either adjusting the RF amplitude, or by adjusting the modulation parameters (for example, duty cycle).

Microwave Frequency: Plasma power can be modulated by changing the operating frequency of the microwave energy. Operating on resonant frequency generates more heat than operating off resonant frequency. In some embodiments, the portion of time spent on resonant frequency vs. off resonant frequency can be utilized to modulate plasma power. In some embodiments, the resonant frequency in the absence of plasma is roughly 2.5 GHz. The actual resonant frequency for a specific chamber is measured for each chamber to account for variance in materials and components. The presence of plasma in the microwave cavity shifts the impedance match of the circuit and therefore may change the frequency that produces maximum power transfer. In some embodiments, the system operates at two frequencies: one before and one after plasma initiation.

Plasma Pulse Duty Cycle: For one embodiment, the plasma pulse modulation frequency is typically between 2 and 18 kHz. For a given system, the lower limit is chosen as a level below which there are stability issues and/or plasma ignition issues. The upper limit is selected at a level beyond which there are diminishing or negligible changes in NO production.

Plasma Duty Cycle: In some embodiments, plasma duty cycle is varied to modulate power within the reactor. Power can be applied to the reactor intermittently, with the plasma pulse duration expressed as a duty cycle ranging from 0 to 100%. These discrete plasma events generate discrete NO boluses that blend as they travel through the system, forming a continuous stream of NO that exits the device. The duty cycle can be continuously varied to generate varying amounts of NO. In cases where the duty cycle adjustment is coarse due to resolution limits, pulse durations can vary between two or more durations pulse by pulse to achieve a target average amount of NO production.

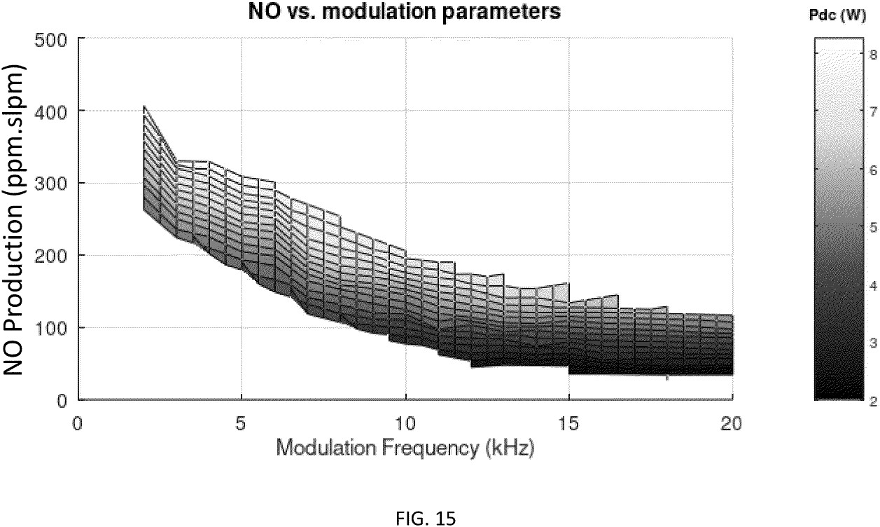

FIG. 15 illustrates an exemplary graph showing NO production values from a system designed to delivery up to 300 ppm.slpm NO. The x-axis represents pulse modulation frequency in kHz. The y axis represents NO production in ppm.slpm. Shading within the graph represents DC power delivered to the plasma. It can be seen that decreases in modulation frequency and increases in pulse power are associated with increased NO production.

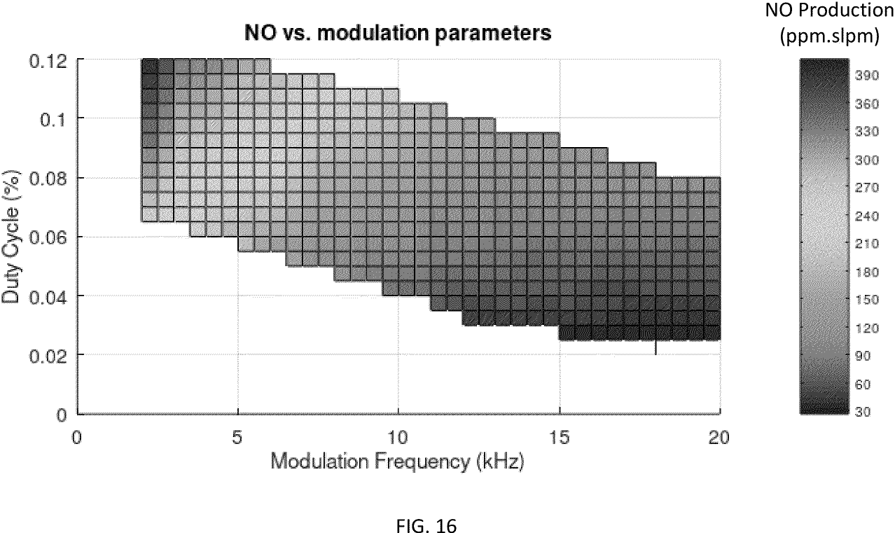

FIG. 16 illustrates an exemplary graph showing NO production values from the same 300 ppm.slpm system as FIG. 15. The x axis represents pulse modulation frequency in kHz. The y axis represents duty cycle in %. The colors in the graph represent the NO production rate in ppm.slpm. This plot demonstrates that there are multiple sets of modulation parameters (combinations of frequency and duty cycle) which are able to generate a particular production number.

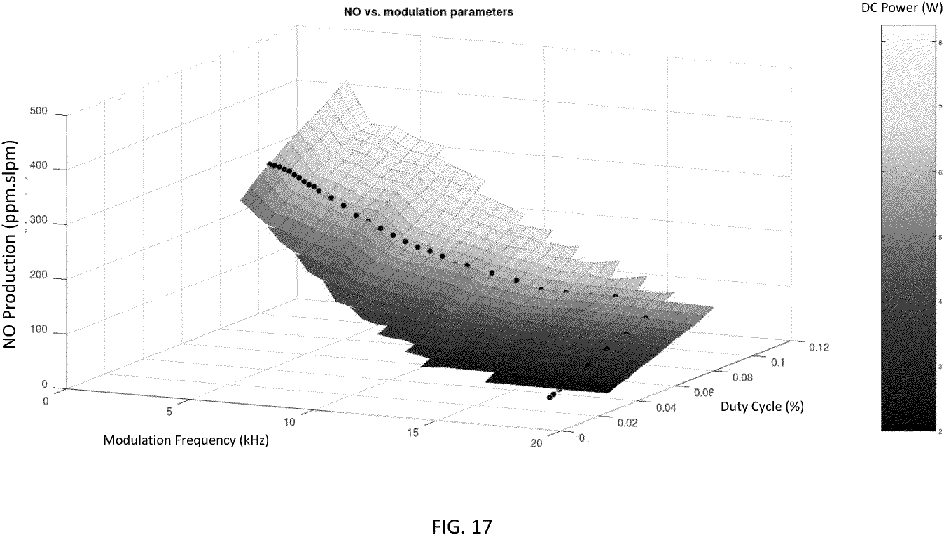

FIG. 17 illustrates an exemplary graph showing all of the power control variables on a single graph. The x axis is pulse modulation frequency in kHz. The y axis is pulse duty cycle in percent and the z axis is NO production in ppm.slpm. Color within the plot is indicative of power delivered to the plasma. A series of black dots on the surface depict a representative combination of settings that could be used to deliver a range of NO production levels. NO production at levels between the discrete production levels can be determined by interpolating between one or more of nearby duty cycle level and frequency level.

The plots in FIG. 15, FIG. 16, and FIG. 17 demonstrate the characteristics of the RF modulation on the NO production, namely, the linear relationship between duty cycle, power, and NO production, and the non-linear inverse relationship between modulation frequency and NO production.

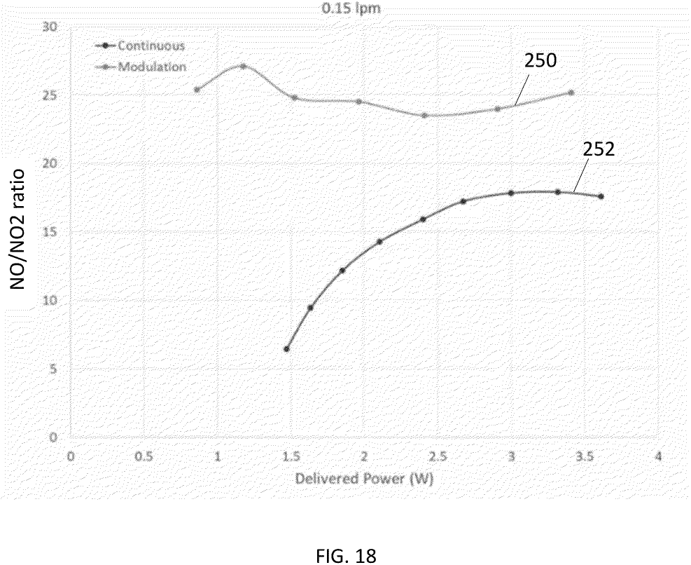

FIG. 18 presents the NO/NO₂ ratio for an exemplary 2.552 GHz microwave NO generator operating in pulsed mode (line 250), for example 800 Hz modulation, and continuous mode (line 252) with 0.15 slpm reactant gas flow. FIG. 18 shows that at all input power levels, pulsed delivery produces a higher NO to NO₂ ratio than continuous power. FIG. 18 also shows that pulsing enables the system to operate at lower delivered power levels than can be sustained by continuous microwave power. This is because the intra-pulse microwave energy is higher during pulsed operation than continuous operation for a given input power level. Although the microwave power is pulsed into the microwave cavity during pulsed operation, the plasma ball is continuous, meaning it does not extinguish and require re-igniting with every pulse. The modulation frequency for similar embodiments can range from roughly 100 Hz up to a few MHz. The lower limit for modulation frequency is governed by the ability to maintain the plasma ball. The upper limit of frequency is governed more by performance limitations of the switching hardware than physical limits of plasma excitation. It will be understood that any frequency can be used, but the size of the cavity and plasma will generally scale as 1/f. For example, lower frequencies will tend toward larger plasmas which require more power. Higher frequencies are associated with smaller, but more power-intense, plasmas. For example, the ISM bands at 915 MHz, 2450 MHz and 5800 MHz can be used.

Low reactant gas pressure is required to initiate the plasma which would be complex and time-consuming to achieve for each microwave pulse. Systems that operate at low reactant gas pressure may be operated at modulation frequencies less than 100 Hz by initiating the plasma ball with each pulse. Benefits can be had from operating a plasma at atmospheric pressures and above, such as increased interaction between reactant gas and plasma for increased NO production and improved production efficiency. Thus, modulation frequencies are typically selected that maintain the plasma ball, enabling production at higher pressures and eliminating the need to partially evacuate the plasma chamber to reignite the plasma for each pulse.

### Dithering

In some embodiments, NO is generated on an as-needed basis following a target NO production profile. In some embodiments, when NO generated matches the target NO production profile, the system makes no change to the operating duty cycle. When NO generated exceeds the target NO production profile, the system turns the duty cycle to zero. When NO generated is below the target NO production profile, the system increases the duty cycle to make more NO. In some embodiments, a NO generation system generates NO in finite boluses. The system tracks the demand for NO and generates a bolus on an as-needed basis when the difference between cumulative NO generated and cumulative NO demand differs by more than the quantity of NO in one bolus. This is effectively discretizing the NO demand curve into NO boluses of the same size. In some embodiments, the NO boluses have differing size.

### Reactant Gas Flow

Reactant Gas Flow Rate: In some embodiments, the reactant gas flow rate is modulated to vary the quantity of gas that interacts with the plasma. Slower flow rates result in less NO formation while faster flow rates result in more NO formation. In some embodiments, the reactant gas flow rate through the reaction chamber is modulated to vary NO generation.

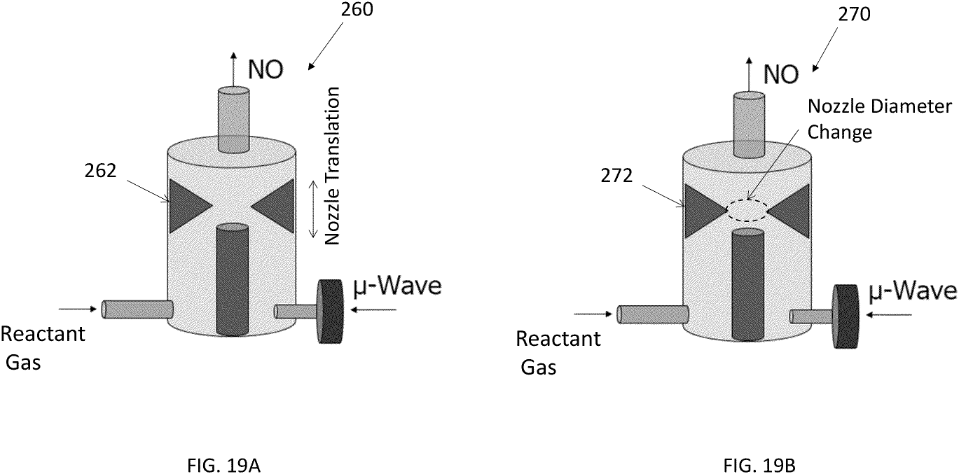

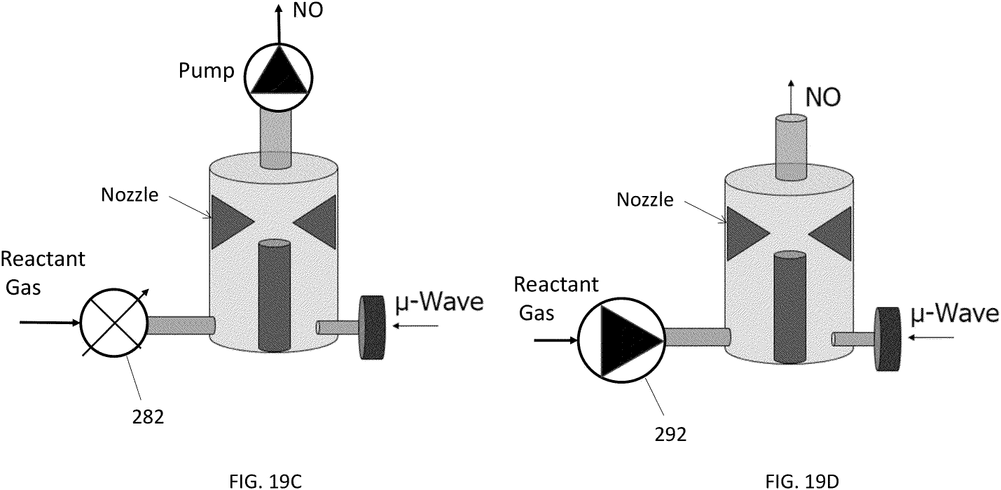

Reactant Gas Pressure: Reactant gas pressure at the plasma affects the quantity of NO formed. In some embodiments, the pressure within the reaction chamber is modulated to vary NO production. In some embodiments, the pressure at the location of the plasma is varied using one or more of the following techniques: varying a nozzle 262 location in a microwave cavity 260 with respect to the plasma as shown FIG. 19A, varying a 272 nozzle shape in a microwave cavity 270 as shown in FIG. 19B, modulating inlet flow controller restriction, for example using a pump 282 with a variable orifice so that the pump pulls down the pressure within the plasma chamber more as shown in FIG. 19C, modulating mass flow rate of reactant gas by one or more of varying pump speed of a pump 292 as shown in FIG. 19D and/or varying the settings of a mass flow controller that receives reactant gas from a pressurized source. An example of varying nozzle shape is an iris design where the inner diameter of the nozzle can be varied. As the nozzle inner diameter gets smaller, the reactant gas flow is required to bend more to the center of the chamber, inducing greater gas/plasma interaction. It should be noted that in some embodiments the plasma chamber and microwave cavity are not in fluid communication. In this case, the pressure within the plasma chamber and the microwave cavity can differ. It should be noted that these different approaches can be used individually or in combination. Each of them vary the interaction between the reactant gas and plasma. Pressure provides more reactant gas molecules in the vicinity of the plasma. Increased reactant gas flow rate provides greater turn-over of reactant gas molecules in the vicinity of the plasma. Slow reactant gas flow rate results in higher temperatures imparted on the reactant gas for longer time, albeit with longer transit time which can result in higher NO oxidation.

Gas/Plasma Interaction: Another contributor to reaction chamber design is the level of gas/plasma interaction. This is essentially the flow profile of reactant gas in the vicinity of the plasma ball. It can affect a variety of performance factors including the stability of the plasma, the production range and the NO/NO₂ ratio. Ideal flow profiles are typically developed computationally with final tuning on the bench. It should be understood that flow that interacts too much with the plasma could extinguish the plasma or make it move in unpredictable ways while gas that does not interact enough with the plasma will be limited in NO production. Ideal interaction of reactant gas with the plasma ball involves having a uniform, symmetrical, thin layer of reactant gas passing around the periphery of the plasma ball to provide enough energy to disassociate N₂ and O₂ molecules without over-heating the reactant gas. Over-heating the reactant gas can form other compounds and result in inefficient production, wasted electrical power and excess heat that the rest of the system will need to accommodate.

The flow profile consists of the shape, velocity, thickness, and proximity of the flow to the plasma. In some embodiments, the flow profile is defined by the inner chamber walls and inlet stub geometry. In some embodiments, an additional nozzle component is utilized to shape the reactant gas flow in the vicinity of the plasma ball. In some embodiments, one or more of the nozzle shape, nozzle location with respect to the plasma, the velocity of reactant gas, the mass flow rate of the reactant gas and thickness of reactant gas flow in the vicinity of the plasma ball can be varied in real time to modulate NO production. Each of these controls affect the temperature change imparted by the plasma to the reactant gas. In some embodiments, the gas flow profile near the plasma is varied during operation to vary the exposure time (flow rate) and the thickness of reactant gas (temperature gradient) to modulate NO production. The flow of the reactant gas through the plasma chamber follows the path of least resistance. In some embodiments, the reactant gas flow changes from laminar to turbulent as a function of the mass flow rate. This affects the level of mixing and interaction of reactant gas with the plasma. In one embodiment, a nozzle downstream of the plasma is moved toward the plasma from a location distant from the plasma (FIG. 19A). As the nozzle is moved toward the plasma, the reactant gas flow path moves closer to the plasma, creating more plasma/gas interaction and increasing the amount of heat transferred to the gas.

### Low NO Production Methods

Low production levels of NO can be produced with a microwave NO generator by modulating one or both of the energy within the plasma and the amount of gas/plasma interaction. Methods to change the plasma energy include one or more of changing the microwave amplitude, pulse frequency, pulse duty cycle, pulse dithering, and microwave frequency (proximity to resonance, for example) and pulse power level. Low production can also be achieved by decreasing the amount of gas/plasma interaction by one or more of decreasing reactant gas mass flow, decreasing the reactant gas density, and decreasing gas/plasma interaction (e.g., directing reactant gas away from the plasma). Some of these approaches to low NO production levels are associated with decreases in power efficiency; however, even inefficient operation when NO production is low requires a low level of power.

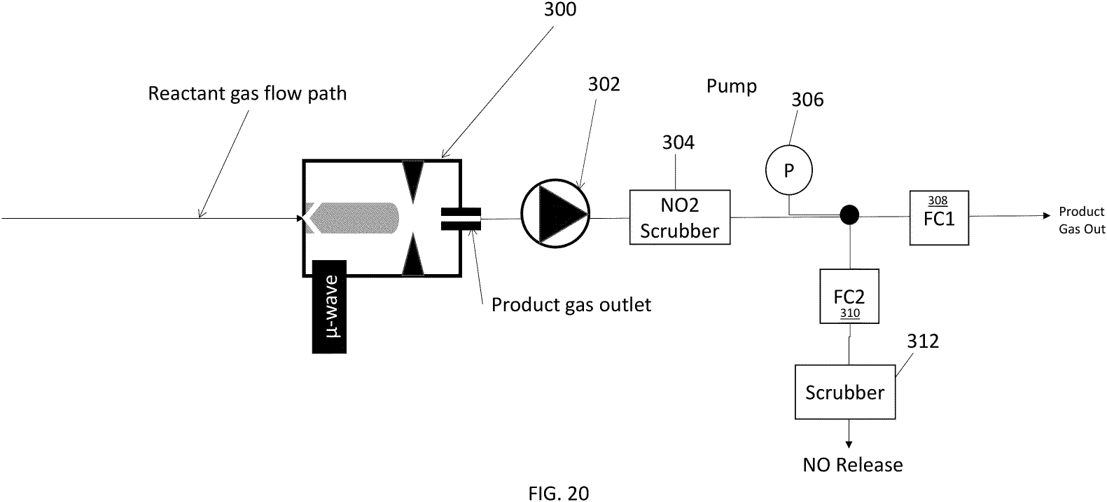

In some embodiments, NO production and NO delivery to the patient are independently controlled. The target amount of NO is delivered to the patient inspiratory limb. Excess NO produced is vented from the system to atmosphere or house vacuum. In some embodiments, the product gas is scrubbed of one or more of NO and NO2 prior to release from the system. FIG. 20 depicts an embodiment of a system where the product gas exiting a combination of a microwave cavity and a plasma chamber 300 passes through a pump 302 and a NO2 scrubber 304. Pressure of the product gas is measured by a pressure sensor 306 in communication with the product gas. A first flow controller 308 controls the amount of product gas sent to the patient. In some embodiments, the amount of NO is proportional to the amount of inspiratory flow. A second flow controller 310 permits the balance of product gas to flow through another scrubber 312 (e.g., NOx scrubber) and out of the system. This allows for continuous plasma chamber operation. In some embodiments, plasma chamber operation (power, frequency, duty cycle) and reactant gas flow rate are constant for greater repeatability.

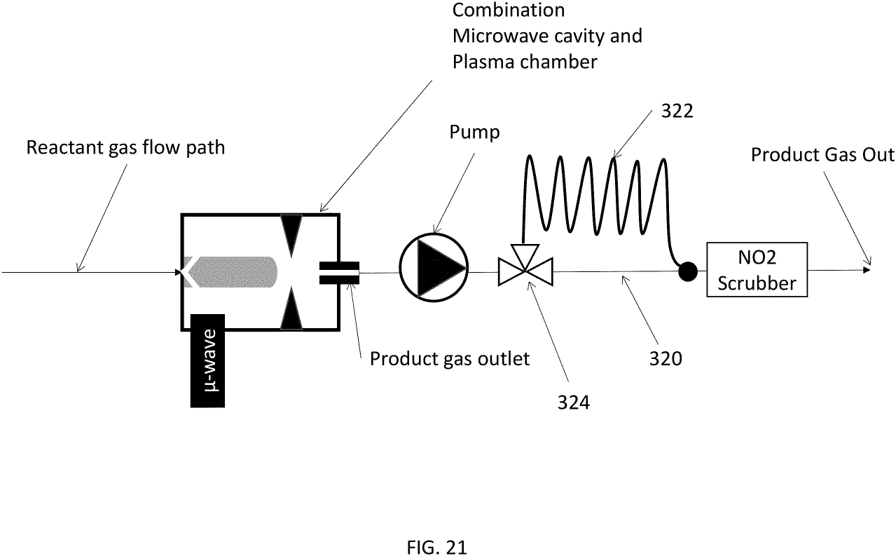

It is also possible to adjust the amount of NO in the product gas after NO production. In some embodiments, NO product gas is permitted to age for an amount of time to oxidize into NO2. Then, the product gas is scrubbed for NO2 resulting in a product gas that has a lower concentration of NO than can be made within the plasma chamber. FIG. 21 depicts an embodiment of an NO generation and/or delivery system that includes first and second product gas pathways: a short pathway 320 and a long pathway 322. In some embodiments, the system selects one or the other path depending on the range of NO production required by the treatment. It will be understood that more than two pathways can be present in the system to provide options of NO concentration in each pathway. In some embodiments, one or more valves, such as a 3-way valve 324, can vary the proportion of product gas flowing through the pathways to provide a continuous range of NO concentrations exiting the system. In some embodiments, there is one product gas path that is purposely long for the loss of product gas NO. This broadens the NO delivery capability of a system by utilizing the long product gas path for low NO demand and short NO path for high demand applications. The control of product gas flow is controlled by the device controller. The flow rate of gas flowing through each path is determined by the controller in multiple ways. In some embodiments, a NO sensor (not shown) measures the product gas concentration and serves as input to a control algorithm. In some embodiments, a NO system is configured to set the valve or valves to specific positions for specific NO output levels. In some embodiments, a controller calculates the amount of NO expected to remain in the product gas based on one or more of the product gas concentration, product gas pressure, product gas flow rate though a path, and volume of the path.

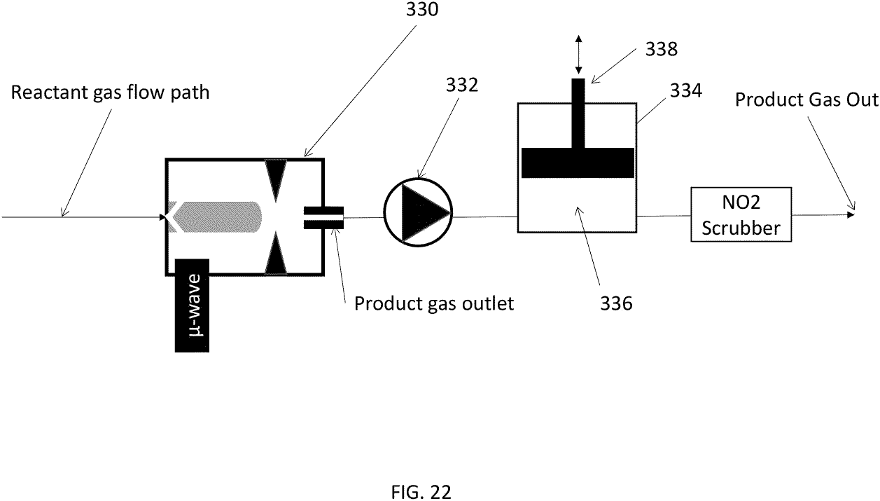

In some embodiments, transit time of product gas is modulated by changing the volume of a reservoir in the system. For short transit times, the volume is small and for longer transit times, the volume is larger. FIG. 22 depicts a system having a combination of a microwave cavity and a plasma chamber 330, a pump 332 with a variable-volume aging reservoir 334. The amount of and pressure of an aging volume 336 of product gas can be adjusted with a movable piston 338.

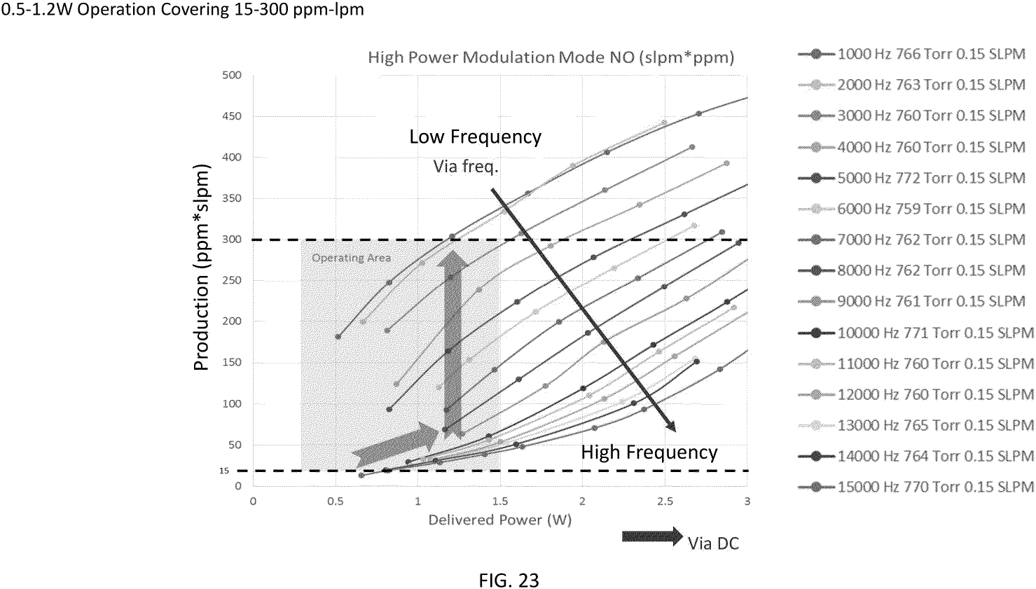

FIG. 23 illustrates an exemplary graph showing the performance of an embodiment of a microwave NO generation device. The Y axis represents NO production in ppm*slpm. The X axis represents input power in Watts. A shaded region extending from 15 to 300 ppm*slpm and from 0.25 to 1.5 W represents an operating window for a particular embodiment. Each curve represents the performance of the system at a particular operating frequency sweeping through duty cycles. The full production range is achieved from low to high by operating at an initial frequency (e.g., 15 kHz) and increasing duty cycle (i.e., pulse duration) to a transition point where duty cycle is held fixed, and frequency is decreased to reach peak NO production levels. In some embodiments, the effect that dominates the modulation frequency vs. production relationship is the effect on the temperature/time profile and its effect on the reaction occurring in the cavity. Thus, slight changes in load impedance and therefore how well matched the cavity is to the amplifier is a much smaller secondary effect which works in the opposite direction (i.e., better match yields more delivered power and therefore more NO).

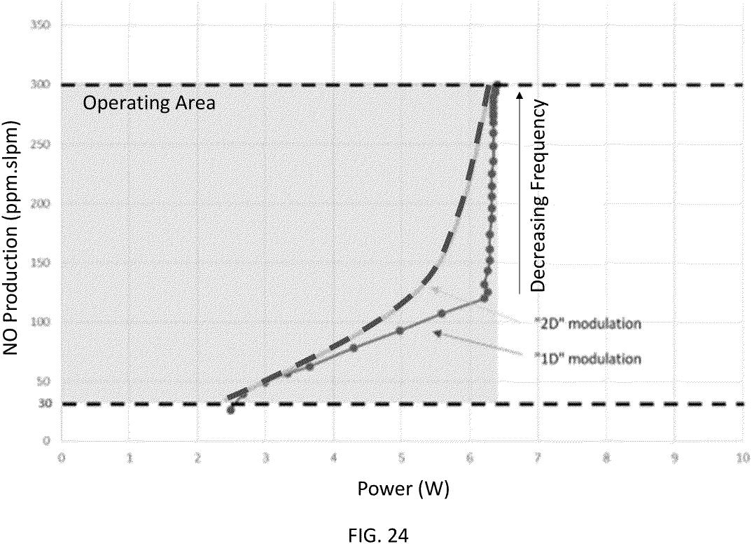

FIG. 24 depicts an exemplary graph showing two approaches to providing a required range of NO production from a microwave plasma ball generator. The lowest NO production level of 30 ppm.slpm is generated at a power level of 2.4 W and a frequency of 18 kHz. As the power is increased from 2.4 W to 6.3 W, the NO production increases linearly from 30 ppm.slpm to 120 ppm.slpm. NO production from 120 ppm.slpm to 300 ppm.slpm is generated by holding the input power constant at 6.3 W and decreasing the frequency. At 300 ppm.slpm of NO production, the frequency is ultimately 2 kHz. This step-wise approach of sequentially adjusting power and then frequency to cover the range of NO production is one way of producing the required range of production (i.e., 1D modulation, as shown).

Each NO production level can actually be achieved by more than one combination of plasma frequency and power. This provides an opportunity to optimize system performance around one or more additional parameters as well. In some embodiments, the frequency and power settings are selected to generate the required amount of NO production with the least amount of power. This can be helpful in systems that are battery operated, for example. Another approach is to maximize NO/NO2 ratio for each NO production level. In some embodiments, the power and frequency for each NO production level is selected to both maximize NO/NO2 ratio and minimize power draw. The dashed line in FIG. 24 depicts an embodiment that modulates frequency and power simultaneously (i.e., 2D modulation) to provide the range of NO production.

In an exemplary application, the NO production is controlled as follows: A patient is prescribed a particular dose level of NO (e.g., mg/hr or inhaled ppm). Based on the prescribed dose, measurements of the treatment (e.g. patient flow rate, flow timing, inspiratory pressure), measured ambient conditions (e.g., ambient pressure, temperature, humidity), the amount of NO expected to be lost between the plasma chamber and the patient (e.g. lost to NO oxidation, and lost to interaction with the scrubber and other parts of the system) and device status (e.g. age of scrubber, type of scrubber, age of electrodes, state of warm-up), the system controller will calculate a quantity of NO to be produced. The system will use that NO production level as an input for an equation or a look up table to determine the power and frequency settings. In some embodiments, the treatment controller determines values in between the values presented in the look-up table by interpolation. In some embodiments, the system utilizes one equation that relates the NO production setting to power and another equation that relates the NO production setting to frequency. For example,

$$\text{P=0} \text{.142Pr+2} \text{.53} \text{and} \text{F=0} \text{.0001Pr\textasciicircum 2-0} \text{.94*Pr+20} \text{.95}$$

where Pr is the desired production level in ppm.slpm, P = the power setting in Watts, and F = the plasma pulse frequency in kHz.

In some embodiments, a plasma ball generator is utilized for a fraction of time within a gas flow. A reservoir is filled with a mixture of NO product gas and non-NO containing gas (e.g., air, N2) so that the product gas concentration is diluted. This enables the device to deliver lower amounts of NO than can be produced within the product gas alone.

In some embodiments, a microwave plasma ball generator includes two or more coupling antennas. A first antenna is utilized to excite the reactant gas and ignite a plasma ball within the plasma chamber. A second antenna is utilized to sustain the plasma ball. By having two different antennas, each can be optimized for particular system conditions (e.g., reactant gas properties, plasma states (on/off). In some embodiments, a microwave NO generator dithers between two or more antennas to modulate NO production. For example, a NO generator can produce levels of NO between that of an inefficient antenna and that of an efficient antenna to achieve production levels between that of each of the antennas. This approach enables a NO microwave NO generation system to sustain a plasma ball while producing a wider range of NO production levels. In some embodiments, each antenna is driven by a separate microwave amplifier circuit. In some embodiments, one or more of the RF frequencies, amplitudes, pulse frequencies, or pulse duty cycles is different for the different antennas. It will be understood that more than two antennas can be used if needed.

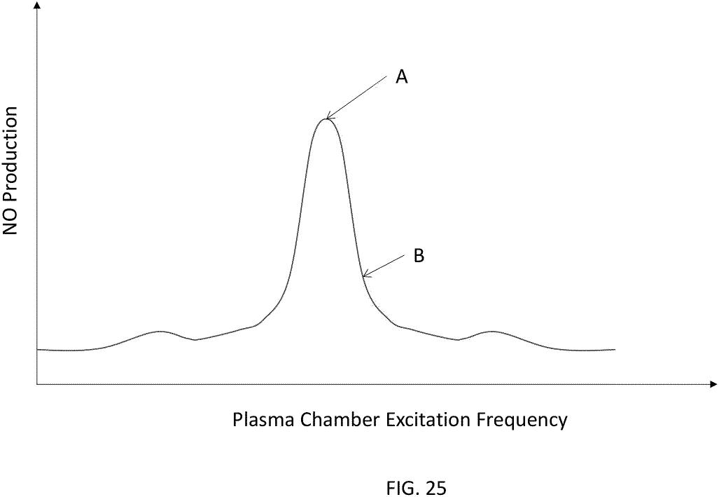

In some embodiments, a microwave NO generation system is able to compensate for changes in impedance between the conditions with and without the plasma ball. The impedance match and power transfer for a given frequency shifts when the plasma chamber changes from no plasma to having a plasma ball. In some embodiments, the NO generation controller initially excites the plasma chamber at or near the resonant frequency of the plasma chamber when no plasma ball is present. When the plasma ball forms, the system shifts the operating frequency to a value at or near the resonant frequency of the system when the plasma ball is present. In some embodiments, a sweep of excitation frequencies reveals the resonant frequency to be a narrow peak. In systems with a narrow resonant peak, more stable, albeit somewhat less power efficient, operation can be had by operating at a frequency near the resonant frequency, rather than exactly at the resonant frequency. FIG. 25 depicts an exemplary graph showing the performance of a NO generation system for a spectrum of excitation frequencies. Operating at the resonant frequency (point A) provides a significant increase in NO output, however small shifts in excitation frequency or resonant frequency can result in large changes in NO production. Assuming an equal amount of time spent at frequency above and below the resonant frequency, the system will underproduce NO since production decreases to the right and to the left of Point A. By operating at point B in FIG. 25, the system produces less NO for a given duty cycle; however, the NO output is more stable in the presence of fluctuations in excitation frequency and resonant frequency. This is because the slope of the curve (change in production over change in frequency) is less at point B. Stable NO output is important for achieving accurate NO dose delivery.

In some embodiments, the oxygen content within the reactant gas is lowered to decrease NO production. In some embodiments, this is done during all operation of the device. In some embodiments, the O2/N2 ratio in the reactant gas is only altered during periods when NO production is required. In some embodiments, the microwave NO generator is either integrated with or embedded in an oxygen concentrator. The reactant gas O2/N2 ratio can be variably adjusted by adjusting the blended amounts of O2 and N2 gas emitted from the oxygen concentrator.

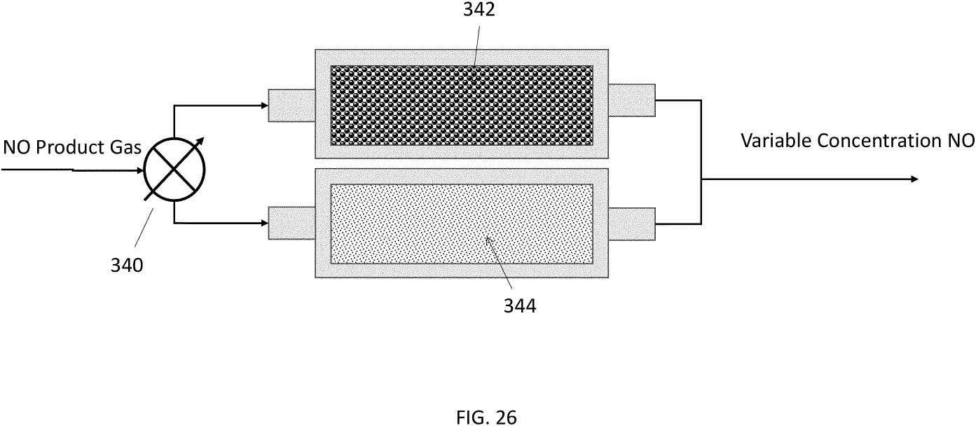

Another method of generating low levels of NO with a microwave NO generator is to partially scrub the product gas for NO. FIG. 26 depicts an exemplary embodiment of a scrubbing portion of a NO generation device. A 3-way valve 340 variably sends product gas to a NOx scrubber 342 and a NO2 scrubber 344. The NOx scrubber 342 (e.g., activated carbon, potassium permanganate) removes NO and NO2. In some embodiments, the NOx scrubber 342 includes an NO scrubber in series with an NO2 scrubber. The NO2 scrubber 344 (e.g., soda lime, ascorbic acid) receives the balance of the flow. In some embodiments, the portion of gas flowing through either leg is entirely variable so long as the sum of flow through each equals the product gas flow rate. In other embodiments, flow is controlled through each leg with a binary valve. Low levels of dilution can be achieved by altering the flow path over time so that the desired portion of product gas is scrubbed for NO. This concept of a split scrubber can operate with continuous product gas flow and discontinuous product gas flow, as found when releasing product gas from a pressurized scrubber architecture.

### NO/NO₂ Ratio Controls

Conventional electrical discharge NO generators produce NO with a NO/NO₂ ratio of 5:1 to 50:1 with 10:1 being typical. Lower ratios result in greater quantities of NO₂ that must be scrubbed for a given amount of NO produced. Higher ratios can result in prolonged antenna, stub, cavity and chamber life due to fewer plasma pulses required to generate a given amount of NO. This reduces the UV exposure and thermal cycling on device components. Higher NO/NO₂ ratios also result in less scrubber mass/volume required to remove the NO₂ produced, and less humidity controls (e.g., desiccant), thereby reducing overall product mass/volume and improving the service life of disposable scrubber components.

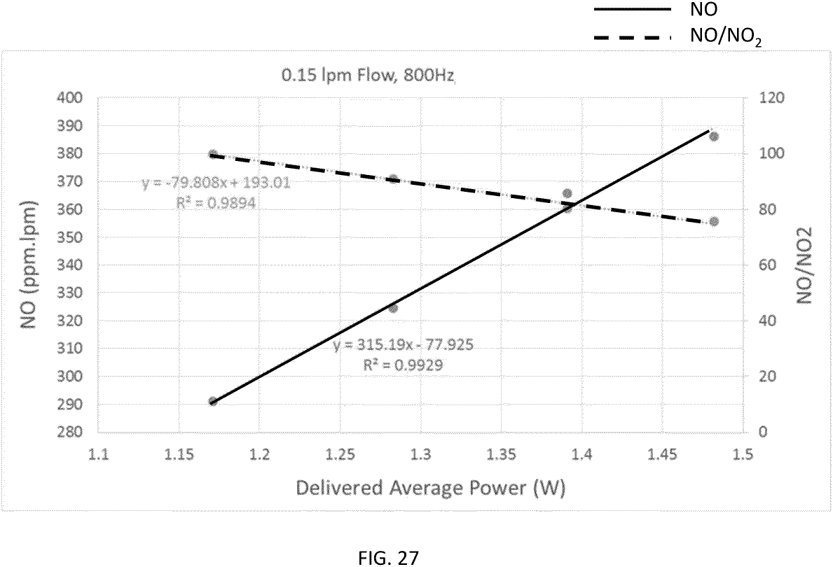

With a microwave plasma NO generation device, the production control parameters that can have an effect on NO/NO₂ ratio include: microwave power, and reactant gas flow rate. FIG. 27 presents the performance of an exemplary microwave plasma NO generator operating at 150 ml/min reactant gas flow and 800 Hz. NO production increases with power. NO/NO₂ ratio is greater than 70 for all power levels evaluated. Other microwave NO systems provide NO/NO₂ ratios of >30 for their entire production range.

### Plasma Power Measurement

Some embodiments of a microwave NO generation device include a power sensor. The power sensor enables measurement of RF/microwave power signals by providing a DC output voltage that is proportional to the mean RF power at the input pin. In one embodiment, a 2.7 nH inductor in series with a 100 pF capacitor provide both DC blocking and input matching. Due to the high peak to average power ratio, a large filter capacitor must be used. This filter capacitance is in parallel with an internal 27 pF capacitor, and a resistance which varies with signal level from 2000 to 500 Ohms. To ensure adequate filtering for RF modulation frequencies as low as 2 kHz, the filter cutoff frequency should be at ~1/20 the minimum input frequency, or 100 Hz. This yields a total capacitance value of 0.8-3.2 uF depending on the resistance. A filter capacitance of 3.3 uF should therefore be sufficient for all signal levels. Capacitance can be increased further at the expense of signal response time.

Light is emitted as atomic particles release energy as they cool down from an excited state exiting the plasma ball. The amount of light emitted is related to the amount of energy in the plasma, which is related to the amount of NO generation. Thus, a calibration function can be derived that relates light output to NO generation. In some embodiments, the relationship between optical output and NO generation is also a function of reactant gas flow rate. In some embodiments, a light intensity sensor receives light from the reaction chamber and transduces that signal into an analog or digital signal indicating the level of plasma power. By measuring actual power within the plasma (vs. power inputs), this approach provides the true power within the plasma, inclusive of any temperature, pressure, and humidity effects on NO production. This measurement is also inclusive of any changes to the hardware (e.g., chamber, stubs, antenna, RF generator) that could affect the power to NO generation relationship over the service life of the generator. In some embodiments, the device controller uses the measurements from the light sensor as an input into a closed-loop control algorithm that targets a specific level of NO production. For example, one embodiment of a NO generator utilizes a look-up table that relates light levels from the plasma with NO production levels to set an initial plasma energy level and reactant gas flow rate. In some embodiments, a controller uses a calibration equation derived from system characterization to relate optical output from the plasma, as measured by the light sensor, to NO production. The NO generator receives a target NO production level from a User or calculates one based on NO demand, as measured by sensors monitoring the patient and/or patient treatment. The NO generator varies one or more of the plasma energy, plasma pulse characteristics or reactant gas flow characteristics from the look-up-table settings to achieve a target NO production level, as measured by the light sensor (e.g., a photodiode). For example, if the light sensor values indicate a NO production level is 50 ppm.lpm and the desired production level is 60 ppm.lpm, the treatment controller can adjust on of the microwave power controls (e.g., pulse duty cycle) to increase NO production in a closed-loop way. In one embodiment, the treatment controller uses proportional integration & derivative (PID) or equivalent approaches to achieve a target NO production level within a flow of reactant gas.

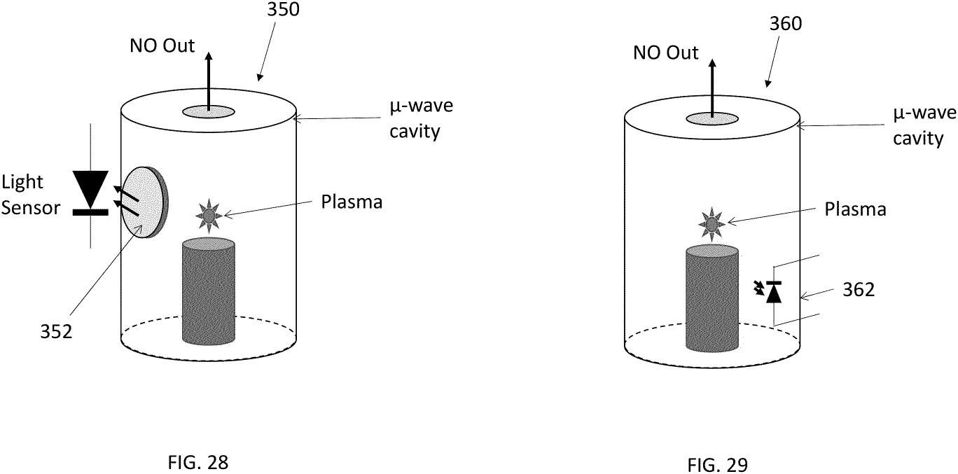

FIG. 28 depicts an exemplary embodiment of a microwave cavity 350 with a window 352 in the wall of the cavity. UV and/or optical light generated by the plasma passes through the window to an optical sensor that transduces light levels to an electrical signal (e.g., voltage, current). FIG. 29 depicts an exemplary embodiment of a microwave cavity 360 with an internal light sensor 362. In some embodiments, the light sensor is upstream of the plasma, as shown. This can protect the light sensor from any sputtered stub materials. Any build-up of materials on the light sensor could affect its sensitivity to light over time, requiring sensor replacement, recalibration, and/or compensation. In some embodiments, the light sensor is closer to the plasma for a stronger light signal. In embodiments that include a UV source to facilitate plasma initiation, the UV source can be used to check the calibration of an optical sensor. In some embodiments, a 240 nm UV lamp shines into a plasma chamber through a window (e.g., quartz window). In some embodiments, a quartz optical fiber is utilized to bring UV light from a UV source to a plasma chamber. In some embodiments, the UV source is located within the plasma chamber. In embodiments that utilize a UV sensor to detect plasma and monitor NO production, the UV sensor can also be used to confirm that the UV source is functioning. The reverse is also true where the UV source can be utilized to confirm functionality of the UV sensor. For example, it could all be implemented as threshold checks for a particular amount of light output expected for a given condition. For example, the UV LED will generate a known amount of signal from the photodiode, which can be checked when it is turned on to make sure it is working properly. A lit plasma will generate a known amount of photodiode signal, which can be checked after the ignition sequence is performed to confirm the plasma is lit.

### Nozzle Design

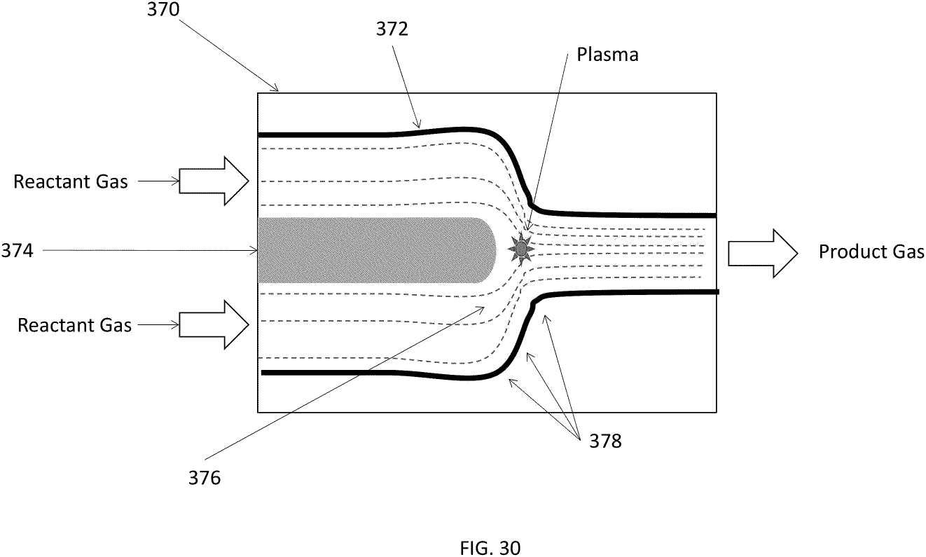

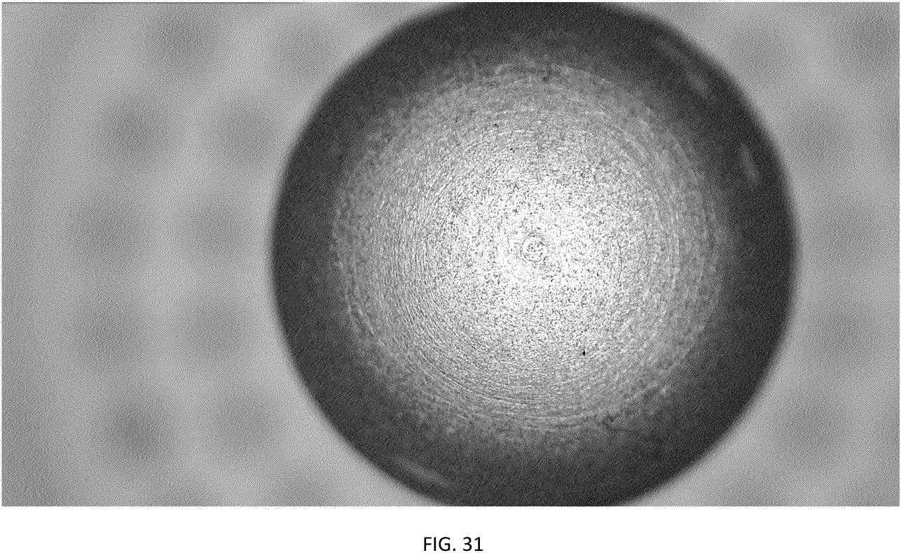

FIG. 30 depicts an exemplary flow profile of reactant gas as it passes through the path defined by the gas flow guides 372 and inlet stub 374 in a microwave cavity 370. The flow guide is located within the microwave chamber and forms a reaction chamber 376. In some embodiments, the microwave cavity 370 and reaction chamber 376 are in fluid communication with each other. In some embodiments, the microwave chamber and the reaction chamber are not in fluid communication.

Reactant gas enters the microwave cavity 370 and travels parallel and on all sides of the inlet stub within the reaction chamber 376. When the gas reaches the end of the inlet stub 374, the radius of the pin is sharper than the flow of gas can turn, creating a local low-pressure zone. The flow guide diameter decreases in the vicinity of the end of the inlet stub, forming a nozzle 378. The nozzle shapes the flow of the reactant gas and increases the velocity of the reactant gas in the vicinity of the plasma. It can be understood by someone familiar with fluid dynamics that the nozzle shape affects the pressure and velocity of reactant gas near the plasma. NO production scales with reactant gas pressure and mass flow rate (more molecules to convert) and is inversely related to reactant gas velocity (less plasma-gas interaction time). At low flow rates, increased transit time can result in more NO oxidation and less net NO out of the system. The location of the plasma ball is dictated by the location of the microwave energy convergence based on the microwave chamber design and microwave energy frequency. The level of gas-plasma interaction is dictated by the pressure, mass flow, and velocity of reactant gas at the plasma.

In some embodiments, the reactant gas is directed in a certain way around the plasma to maximize NO, minimize NO2, minimize power, and minimize interaction between the plasma and the stub to maximize stub life. For example, a design utilizing a downstream nozzle such as shown in FIG. 19A, can decrease reactant gas/plasma interaction by moving the nozzle/flow guide away from the plasma ball. In some embodiments, these features are optimized for NO production with minimal NO₂ production. High NO:NO₂ ratio decreases overall energy requirements, enabling smaller, lighter batteries and decreases the amount of NO2 scrubbing material required while increasing scrubber longevity.

In some embodiments, the flow guide is a straight tube. In some embodiments, the flow guide is tubular with varying inner diameter that necks down downstream of the plasma ball. In other embodiments, the flow guide consists of a non-conductive annular shape located within a tubular structure.

A gap is maintained between the plasma ball and the inlet stub. This gap, in concert with convective and conduction cooling of the inlet stub from reactant gas flow and surrounding components, enables the NO generation system to keep inlet stub surface temperatures sufficiently low to minimize long term thermal damage to the inlet stub. FIG. 31 is an exemplary photograph of the end of a 4 mm diameter aluminum inlet stub after multiple hours of NO generation. Although the inlet stub is made from a relatively low melting point metal (660° C. compared to 2446° C. for Iridium, a typical plasma electrode material for electrical discharges), the stub shows no signs of wear or thermal damage from the plasma ball. This enables the use of less expensive, lighter, and easier to machine aluminum for stub fabrication.

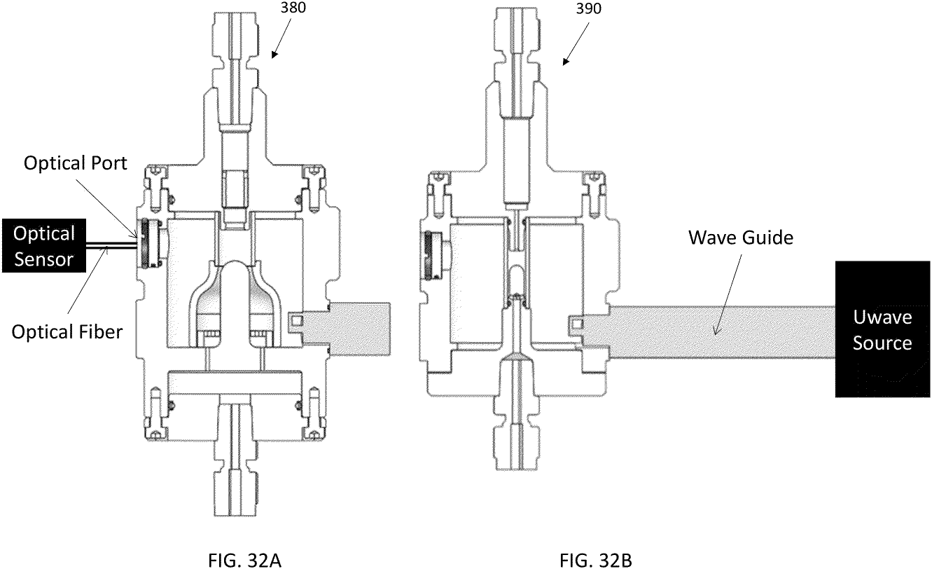

FIG. 32A and FIG. 32B depict two embodiments of reactant gas flow path designs through a microwave cavity. FIG. 32A illustrates a microwave cavity 380 that utilizes a two-piece flow guide with a translucent cylindrical section at the top for observation and/or optical power measurement. Optical measurements can also be used to confirm plasma ball formation and quantify plasma formation time. Reactant gas flow enters the bottom of the image and necks down in the region of the end of the inlet stub. The inlet stub is mechanically and electrically connected to the outer microwave cavity.

FIG. 32B depicts an exemplary microwave cavity 390 where reactant gas is introduced to the system through the inlet stub. The flow guide consists of a straight cylinder. In some embodiments, the straight cylinder is translucent (e.g., quartz) to enable visualization of the plasma. In some embodiments, an optical sensor is utilized to detect the presence, absence and/or energy level of the plasma ball. FIG. 32A depicts an optical sensor located outside the microwave cavity that receives light from the plasma ball. In some embodiments, the light sensor is mounted directly to the microwave cavity. In some embodimentss, as shown, the light sensor is mounted remotely and receives plasma ball light output through a length of optical fiber.

The microwave energy source in FIG. 32B is remote from the microwave cavity and connected by a waveguide. This approach provides alternatives to packaging a microwave NO generator within a larger system and is applicable to all embodiments presented herein.

### Reactant Gas

Reactant gas for a microwave NO generation device includes nitrogen and oxygen at a minimum. In some embodiments, atmospheric air with 21% oxygen is used. In some embodiments, a Stoichiometric ratio of 50% oxygen and 50% nitrogen to maximize NO production is used. In some embodiments, the reactant gas has a lower amount of oxygen (e.g., 1-20%) to decrease the amount of NO produced. Other ratios of oxygen to nitrogen have also been contemplated.

The microwave NO generation device can operate at various pressures of reactant gas, including levels below atmospheric pressure to well above atmospheric pressure. In some embodiments, the pressure of the reactant gas is below atmospheric (vacuum) to assist initiation of plasma. In some embodiments, the microwave NO generator operates at below atmospheric pressure all of the time to improve resolution and low-end range of NO production. In some embodiments, a system intended to generate NO at altitudes up to 3000 m is designed to operate with 10.2 psi reactant gas pressure. A variable flow restriction before the plasma chamber makes the plasma chamber pressure the same for all lower altitudes. This causes the NO production rate to be less sensitive to changes in atmospheric pressure. In some embodiments, the device controller varies the variable flow restriction based on ambient pressure measurements made by a pressure sensor in the device to target a specific plasma chamber pressure. In some embodiments, the relationship between flow restriction and chamber pressure can depend on the specific implementation, type of restriction, pump, and inlet air properties (namely pressure). For example, if the inlet air is ambient atmospheric pressure air at sea level, there is a variable valve at the inlet, and the pump is pulling air through the cavity, reducing the inlet size (“increasing” the restriction) would reduce the pressure in the cavity. In this configuration, the maximum pressure is atmospheric pressure, as with the pump pulling through the cavity, it would not be possible to pressurize the cavity.

In some embodiments, reactant gas is processed to remove VOCs, NO, NO2, water and particulates prior to NO generation. This minimizes the variation of compounds entering the reaction chamber and limits the quantity and kinds of chemical reactions that can occur within the plasma chamber, thereby limiting the variation of compounds present in the product gas. In some embodiments, the reactant gas scrubber is comprised of one or more of the following materials: soda lime, activated carbon, potassium permanganate, and molecular sieve. Particulate is removed with particle filters. In some embodiments, particle filters are constructed from one or more of the following materials: ptfe, borosilicate glass fiber, cellulose, wire mesh, and polyester. For example, the pore size of the reactant gas particle filter can typically be 20 micron or less. In some embodiments, the reactant gas filter pore size is in the sub-micron range (e.g., 0.22 micron).

In some embodiments, water is either partially or completely removed from the reactant gas before entering the reaction chamber. This can be done by any variety of means. Water within the reactant gas affects NO production in a number of ways, including increasing the thermal mass of the reactant gas (energy required to increase temperature), and increasing the expansion of reactant gas as water turns into steam from the heat of the plasma. Both of these effects decrease NO production for a given amount of energy input as reactant gas water content increases. In some embodiments, water enters the reaction chamber at atmospheric levels. This is acceptable, however the elevated pressure within some NO generation systems will cause condensation within the system under some environmental conditions (high relative humidity). Given the variation of NO production due to reactant gas humidity, some embodiments measure the water content and/or humidity of the reactant gas and adjust the microwave energy accordingly to compensate for the humidity effects. Similar compensation is done for reactant gas pressure and/or temperature in some embodiments. In some embodiments, removal of all or nearly all water from the reactant gas can be achieved so that compensation for reactant gas water content is not required.

### Product Gas Quenching

NO is formed at elevated temperatures. In some embodiments, reactant gas is heated to 2000 to 3000°K to form product gas. Thus, hot plasma is required for efficient NO generation. Microwave and arc-discharge are two examples of hot plasma. In some embodiments, product gas is quenched after NO formation to minimize the potential for NO loss. Nitrogen and oxygen do not react at room temperature. Quenching involves rapidly reducing the temperature of the product gas. It can be done to limit the nitrogen - oxygen reactions that occur at lower temperature (e.g., <2000°K) by spending less time in that temperature range. In other words, quenching can arrest particular chemical reactions, thereby locking in a particular amount of NO and NO₂. The quenching step of the process can be controlled by (1) varying the gap between plasma ball and chamber outlet, (2) varying the distance from plasma to the heat exchanger, (3) thermally coupling the reaction chamber exit nozzle to a heat sink. In some embodiments, they are the same component, and/or (4) actively cooling the exit nozzle and/or heat exchanger with a gas or liquid flow that is independent of and separated from the product gas flow.

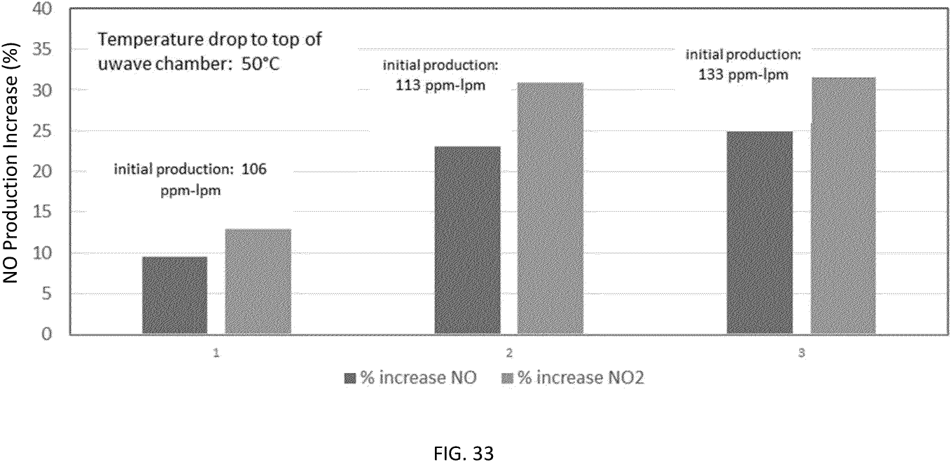

An experimental demonstration of the effects of product gas quenching on NO and NO2 output was performed by actively cooling the gas outlet of the plasma chamber with compressed gas resulting in a 50° C. reduction in outlet temperature, as measured by infrared camera. FIG. 33 illustrates a graph showing the results for three exemplary NO production levels. NO output increased 10% to 25% with no changes in plasma settings or reactant gas flow rate. Similarly, NO2 output increased from 12 to 31% when the gas outlet was actively cooled. This indicates that a greater amount of NOx, in general, was retained when the gas outlet was cooled. Given that the NO to NO2 ratio is typically 10:1 or more, the additional moles of NO output by quenching far outweighs the additional moles of NO2 output by quenching.

### Reactant Gas Cooling

In some embodiments, the reactant gas and or inlet stub are actively cooled. A 50° C. decrease in temperature can increase NO production by 65% with no changes to reactant gas mass flow rate, plasma power or plasma frequency.

### Component Design and Materials Selection

Stubs - The stubs are required to be electrically conductive and are typically metallic. In some embodiments, the stubs are aluminum.

Microwave Cavity: The microwave cavity is typically constructed from an electrically conductive material because it serves as a stub as well as a faraday cage to contain electromagnetic emissions. In some embodiments, the microwave cavity is constructed of aluminum. In some embodiments, the microwave cavity is constructed of a non-electrically conductive material (e.g., plastic, ceramic, glass) and is coated or lined with an electrically conductive material (e.g., aluminum, silver, nickel, copper). In some embodiments, the microwave cavity is made from a reasonably conductive metal (e.g., aluminum) and plated with a highly conductive material (e.g., silver) to increase the conductive skin effect within the chamber.

Antenna: The structure used to couple power into the cavity, referred herein as the “antenna,” can be a loop or probe or an open waveguide depending on the wavelengths and structure sizes involved. The antenna is made from electrically conductive material. In some embodiments, the antenna is a rectangular loop design made from 0.254 mm thick copper measuring 4.75 mm in length, 3.4 mm in height and 2 mm in width. The microwave cavity needs to produce a highly localized electric field and a symmetric field is also a plus for practical reasons. The central stub post in the axisymmetric cavity of some embodiments might also be referred to as an “antenna” by some in the field, however most engineers would not call it this because “antenna” is usually used to describe structures used to launch propagating waves rather than those which only aim to produce a localized field.

Reaction Chamber/Gas flow guide: The gas flow guide is impermeable to gas and permeable to microwaves. Non-metallic materials like quartz, Teflon, glass, and ceramic are typical materials for constructing the gas flow guide. In some embodiments, the flow guide is transparent to ultraviolet light to allow a photon source to ionize the reactant gas and facilitate ignition of the plasma.

Circulator: The match provided by the antenna and cavity is not perfect under all conditions; therefore, there can be operating conditions with significant reflected power (VSWR > 5). In order to protect the power amplifier from damage, a circulator or isolator is placed between the power amplifier output and the load. In some embodiments, a 3-port circulator is utilized with returning energy directed out the 3^{rd} port to a termination resistor. In some embodiments, the circulator includes a built-in termination of the isolator to eliminate the need for an additional termination. This built-in termination can reduce the size of the circulator. Isolation levels of >20 dB have been found to be sufficient. The terminating resistor must be of sufficient quality to be able to handle the power dissipation as well as maintain a good VSWR at the operating frequency.

Directional Coupler: In some embodiments, a directional coupler is implemented to measure the amplifier output power to ensure the system is operating properly. For example, a microstrip directional coupler can be used. In some embodiments, the coupling factor is ~25 dB at 2.5 GHz. The coupling factor for a directional coupler relates to the fraction of the input power that will be coupled to the coupled port. For example, if a directional coupler has a coupling factor of 25 dB, and a signal of 0 dBm is input, then there will be a signal of -25 dBm output at the coupled port. This signal is passed through to a power sensor via an additional PI attenuator to provide additional attenuation if required. Insertion loss is quite low at <0.1 dB. Directivity is excellent at >29 dB above 2.5 GHz. The coupled reflected power is terminated with a 50 Ohm resistor. The coupler properties depend on the dielectric properties of the PCB substrate, as it is a microstrip design. The above coupling parameters assume a PCB fabricated out of Rogers RO4350B material with a thickness of 0.010 inches (10 mils). Only the dielectric between the top layer and the ground plane beneath it must be controlled to these specifications.

### Plasma Initiation

Ignition of microwave plasma requires a certain level of power. The amount of power is less when ions are present in the gap. Thus, continuation of a plasma once initiated is not difficult. There are limits, however, to the plasma ignition as the plasma pulse frequency slows. In one embodiment, for example, there are insufficient ions present for rapid plasma generation after 500 msec. Thus, for plasma pulses that occur at a greater period of time, additional measures may be required to help ignite the plasma. The following list of methods and designs to facilitate initiating plasma can be used on their own or in combination.

In some embodiments, a vacuum is pulled in the plasma chamber to facilitate initial plasma formation. Electrons, which cause breakdown of the gas through ionization, gain more energy between collisions at lower pressures and thus are more effectively ionizing the background air molecules. The lower the pressure, the longer the mean free path or distance between the molecules of air and more time for the electric field to continue to accelerate the electrons and thus give them higher velocity or effectively kinetic energy. The electric field is of course generated in the microwave cavity by the microwave power source. In some embodiments, 7 psi of vacuum (~400 Torr, ½ Atm) is pulled in the chamber. In some embodiments, the level of vacuum falls within a range of 100 mTorr to 500 Torr. In some embodiments, the plasma ball is continuous with the energy within it varying over time. In some systems that operate at or above atmospheric pressure within the reaction chamber, a vacuum is pulled on the reaction chamber to assist in starting/restarting the plasma ball.

In some embodiments, the plasma chamber pressure is maintained at a pressure below atmospheric pressure all the time. This enables plasma initiation whenever desired as well as lower levels of NO production. In some embodiments, a microwave NO generation system will have two or more operating modes that have different chamber pressures and corresponding NO production ranges.

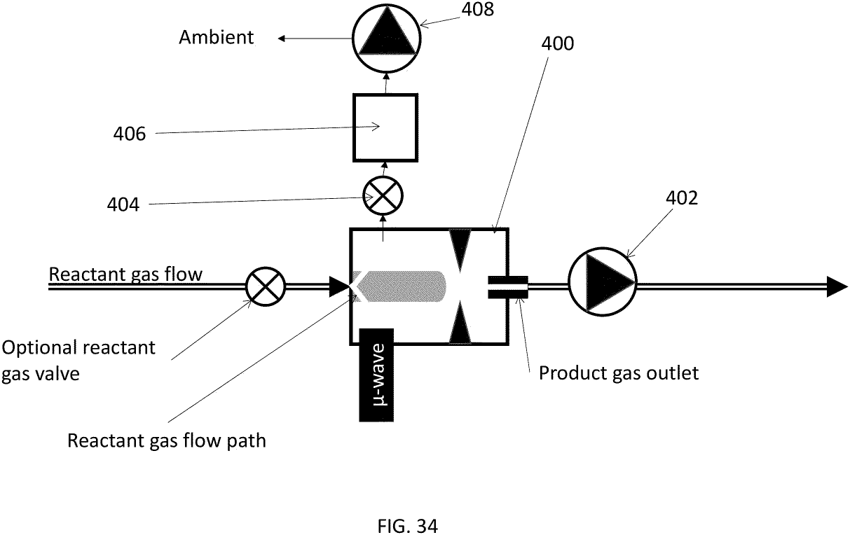

In some embodiments, the plasma chamber pressure is only decreased momentarily to facilitate plasma initiation. FIG. 34 depicts an exemplary system that momentarily lowers the pressure within the plasma chamber to facilitate plasma initiation. Reactant gas enters the combination microwave cavity and plasma chamber 400. A first gas flow path for product gas exits the chamber 400 and passes through a pump 402. A second gas outflow path exits the chamber 400 through a valve 404 (such as a vacuum valve), drawn by a pump 408. When the outflow valve is closed, the pump can draw down the pressure within a reservoir 406, or vacuum chamber, located between the pump 408 and valve 404. When the outflow valve opens, a vacuum within the reservoir as well as the pump flow draw gas out of the plasma chamber to momentarily decrease the pressure within the chamber and facilitate plasma formation when microwave energy is ON. After initiation of the plasma ball, the valve is closed again. The degree and duration of the pressure drop within the plasma chamber is increased in some embodiments by placing a valve in the reactant gas flow path as well (optional reactant gas valve shown). In some embodiments, the reactant gas valve operates out of phase with the vacuum path valve.

Another way to facilitate plasma initiation with microwave energy is to shine UV light into the chamber. The UV light ionizes reactant gas molecules within the chamber, making it easier to create a first breakdown event. In some embodiments, a 1.2 mW UV LED shines light into the plasma chamber through a quartz window in the side of the chamber. In one embodiment, an optical lens is utilized to focus the UV light at the location of the plasma ball. Once plasma is established, the UV light is optional since the plasma generates ions itself, making it self-sustaining when the pulse frequency is above a minimum threshold. In another embodiment, a heated wire is utilized to provide ions to the plasma chamber via thermionic emissions. These approaches to facilitating plasma formation decrease the amount of time required to ignite plasma. They also improve the precision of breakdown time, i.e., the time between turning on the plasma in software and when the plasma actually forms. Improvements in plasma ignition precision enable more accurate generation of NO because the actual duration of each plasma pulse can be known.

In some embodiments, the actual breakdown time of the plasma ignition is measured and compensated for. In some embodiments, a plasma pulse is sustained a variable length of time to accommodate variance in plasma initiation time. For example, if a plasma initiation takes 5 microseconds longer than normal to form, the plasma pulse can be sustained an extra 5 microseconds so that a target amount of NO is generated. In another embodiment, the power within a given plasma pulse is varied based on the initiation time. For example, if plasma initiation for a particular pulse takes 5 microseconds longer than normal, the power within the plasma pulse can be increased to make additional NO within the pulse to stay on target for overall NO production. In some embodiments, deviations in plasma initiation time are compensated for in subsequent plasma pulses by adjusting the frequency and/or duration of plasma pulses.

In some embodiments, a large pulse of energy is applied to enable plasma initiation. This can enable plasma initiation at higher chamber pressures, including atmospheric pressure. This increased energy cost would be acceptable for devices that are powered by a wall power outlet, where energy efficiency is less critical. Once the plasma is initiated, the power to the plasma can be turned down to a lower level for sustaining the plasma ball.

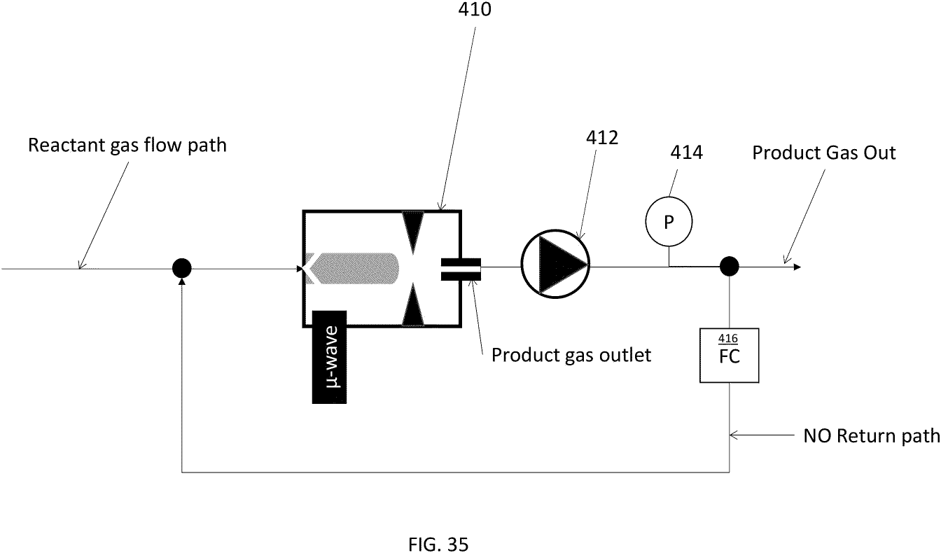

In some embodiments, a stream of product gas containing NO is introduced to the reactant gas flow upstream of the plasma chamber so that the reactive NO can facilitate plasma formation. FIG. 35 depicts an exemplary architecture whereby NO exits a combination microwave cavity and plasma chamber 410 and passes through a pump 412. The pressure of the product gas is measured with a pressure sensor 414. A flow controller 416 provides a return flow of NO to the reactant gas flow prior to the plasma chamber. In some embodiments (not shown), the NO flow is introduced directly to the plasma chamber.

In some embodiments, plasma initiation is facilitated by creating an acoustic wave within the plasma chamber to fluctuate the pressure within the chamber such that plasma can be initiated at a low point in the pressure cycle. In another embodiment, a standing acoustic wave is generated within the plasma chamber with a local minimum located where the plasma ball is generated. In some embodiments, this acoustic wave is only turned on to help ignite the plasma. In one embodiment, the acoustic wave is generated by a diaphragm within the wall of the plasma chamber that acts on the gas within the plasma chamber.

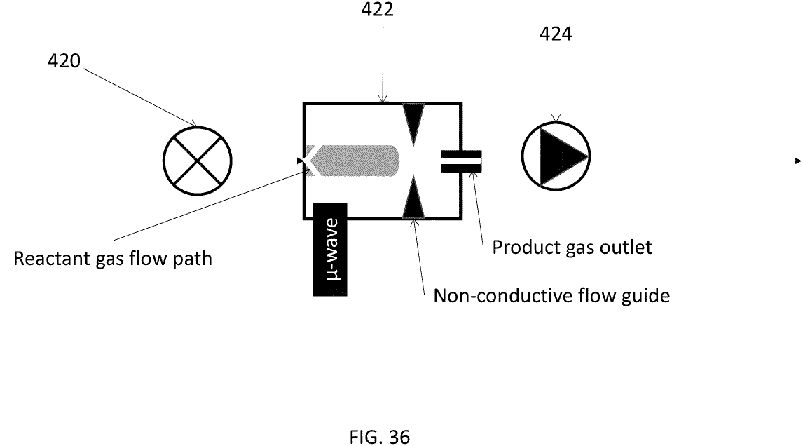

FIG. 36 depicts an embodiment of a NO generator with a flow restriction 420 before the combination microwave cavity and plasma chamber 422. The flow restriction 420 can be passive (e.g., orifice or critical orifice) or active (binary valve, proportional valve, etc.). The flow restriction may be partial or completely obstruct the reactant gas flow. A pump 424 downstream of the plasma chamber pulls reactant gas through the chamber. In some embodiments, the flow restriction is passive, permitting a target reactant gas flow rate under nominal conditions. When the pump is operated faster, the orifice restricts flow to a maximum value and the pressure within the reaction chamber decreases. In some embodiments, the pump is operated at a high flow rate to generate a low pressure within the reaction chamber to initiate plasma activity.

In some embodiments, one or more active flow control devices is located before the reaction chamber. When a system initiates plasma, the flow control device is partially or fully closed and the pump is utilized to pull gas out of the reaction chamber to lower pressure and facilitate initiation of the plasma ball. Examples of active flow control devices include binary valves, proportional valves, needle valves, mass flow controllers, and other type of flow control devices. In some embodiments, the pump operates at a constant flow rate. In some embodiments, the pump changes flow rate to modulate reactant gas flow and pressure within the chamber. When the active flow control device restricts flow and/or completely impedes flow, the pump pulls down the pressure within the reaction chamber.

In some embodiments, a pre-chamber flow control device and/or pump is utilized to modulate pressure within the reaction chamber to compensate for changes in atmospheric pressure as can occur with changes in elevation.

### Architecture

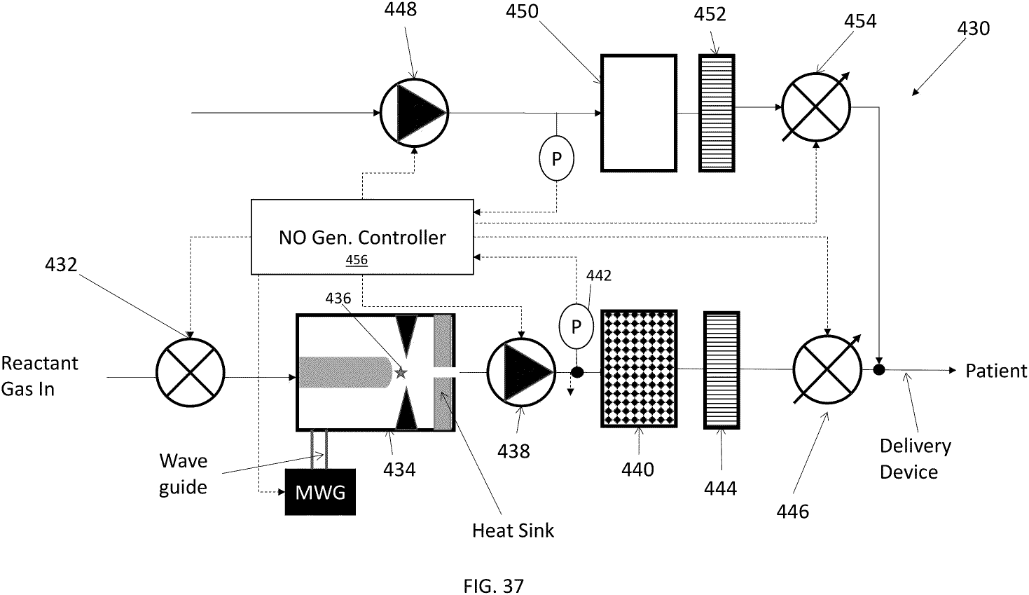

In some embodiments, a microwave plasma ball generator is utilized in a pressurized scrubber architecture, as shown in FIG. 37. Reactant gas is pulled into the system 430 through a flow restriction 432. This generates a pressure within a plasma chamber 434 that is below atmospheric pressure to assist with plasma initiation. Gas passes through the plasma chamber, along the length of the inlet stub and past a plasma ball 436. Gas then passes through an outlet stub on the exit and out to the pump 438. In some embodiments, the outlet stub and corresponding wall of the microwave cavity function as a heat exchanger for cooling product gas.

In some embodiments, the pump operates continuously. In some embodiments, the pump operates intermittently. In some embodiments, intermittent pump operation is governed by the pressure within a scrubber 440, as measured by a pressure sensor 442. For example, the pump turns on when the scrubber pressure is below a threshold (e.g., 10 psi) and turns off when it reaches that threshold.

In some embodiments, plasma operation is continuous with a specific pulse frequency and duty cycle. In some embodiments, plasma operation is intermittent. In some embodiments, the plasma is active long enough to make a target number of moles of NO and then turns off while the pump continues operating until the target pressure is achieved. Reactant gas and NO gas mix within the pressurized scrubber in the time prior to the next patient breath. The target number of moles of NO can be based on one or more of a target dosing rate for a patient, a predicted amount of NO loss expected from oxidation, a predicted amount of NO loss expected from interaction with the scrubber, a target quantity of NO molecules desired for a specific breath, predicted/characterized NO/NO2 ratio.

As NO gas exits the scrubber 440, it passes through a particle filter 444 and a flow controller 446 (e.g., mass flow controller) that controls the flow rate of product gas delivered to the patient through the delivery device.

In some embodiments (not shown), the delivery device is purged with non-NO-containing gas (e.g., reactant gas) between breaths to mitigate against NO₂ formation within the delivery device using a purge pump 448, a purge reservoir 450, a filter 452, and a purge flow controller 454.

In some embodiments, a NO generation controller 456 receives pressure information from the scrubber pressure sensor and the purge reservoir pressure sensor. The NO generation controller also receives a target NO dose from a user interface, external device, or within system memory. The NO generation controller 456 controls one or more of the inlet flow restriction, microwave generator, plasma flow pump, purge flow pump, purge flow controller, and output flow controller.

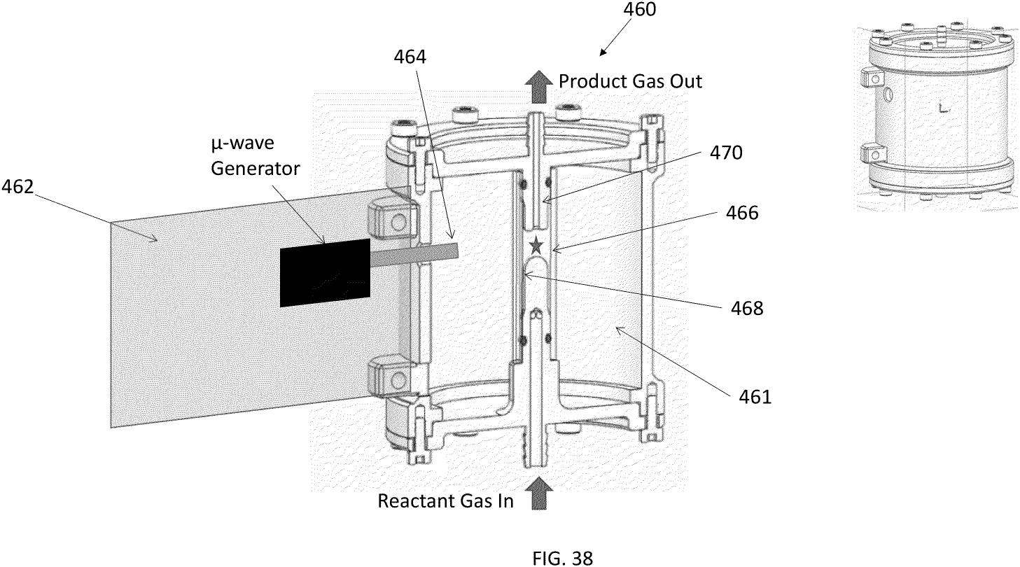

FIG. 38 depicts an embodiment where the process controller and microwave generator are connected directly to the microwave cavity. A cut-away view of a microwave cavity and reaction chamber 460 is shown. The microwave generation and control board 462 can attach to ears on the microwave cavity 461. A microwave source antenna 464 extends into the microwave cavity. A plasma chamber 466 is concentric with the microwave cavity 461 and in fluid isolation (no communication) from the microwave cavity. The flow guide consists of a tube located axially in the design by an inlet stub 468 and an output stub/port 470 and sealed with O-rings. Reactant gas flows from the bottom to the top of the image, entering the plasma chamber radially at a location above the first O-ring (out of this view plane). Reactant gas flows along the outer surface of the inlet stub and then converges towards the center of the structure to exit out of the outlet port. As the reactant gas flows and converges, it tangentially interacts with the plasma ball (depicted as a star), partially converting oxygen and nitrogen in the reactant gas into NO and NO₂.

### NO Generator Performance

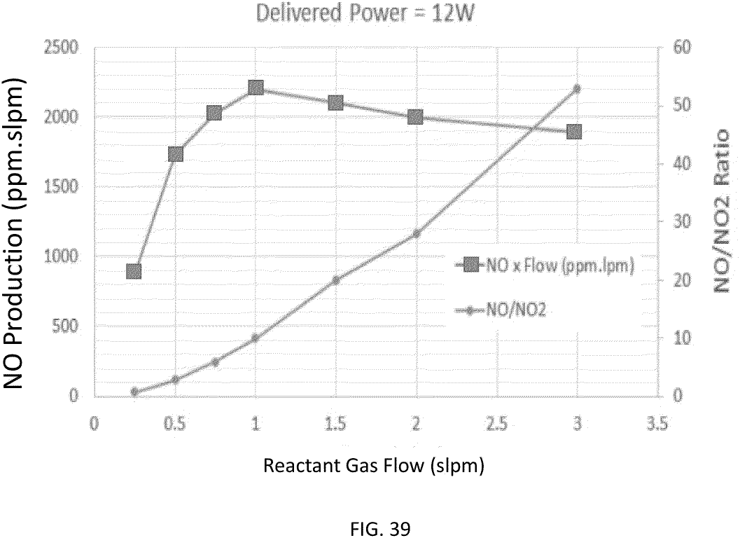

FIG. 39 depicts an exemplary graph showing the performance of a representative NO generator operating at 12 W input power with separate microwave cavity and reaction chamber. The x-axis represents reactant gas flow rate through the reaction chamber. The left axis presents NO production in (ppm.slpm). The right axis represents the NO/NO₂ ratio. In general, higher NO/NO₂ ratio can be used unless the system has a subsequent NO₂ to NO converter.

FIG. 39 shows that NO production in this specific example is fairly independent of reactant gas flow rate at flow rates greater than 0.75 lpm but is dependent on flow rate at reactant gas flow rates less than 0.75 lpm. The NO/NO2 ratio increases proportionally with flow rate. This is likely due to a combination of greater reactant gas / plasma interaction and shorter transit time from the reactor to the NO and NO2 sensor used in the experiment. Longer transit times associated with slower flow rates provide more time for NO to oxidize into NO₂, thereby decreasing the NO/NO₂ ratio. In some embodiments, product gas is diluted soon after the reaction chamber to reduce NO concentration, thereby reducing oxidation rate, and shortening the transit time to the patient. In some embodiments, the dilution consists of nitrogen, oxygen, or a mixture of the two (e.g., air). In some embodiments, helium is used to dilute the NO product gas due to the inertness of helium as well as clinical benefits of heliox treatment.

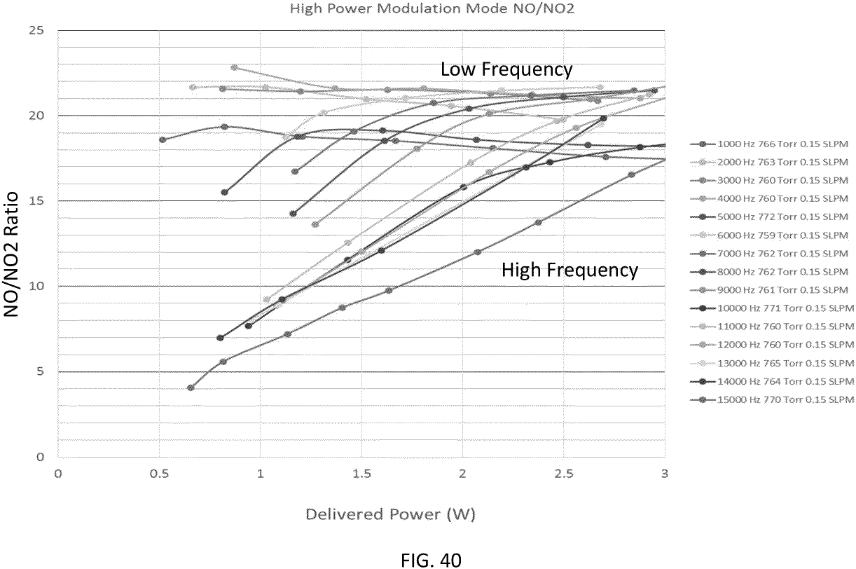

FIG. 40 depicts a graph showing exemplary NO/NO₂ performance for another embodiment of the system for a variety of pulse frequencies and duty cycles. Curves represent sweeps of duty cycle for a specific pulse frequency. The NO/NO₂ ratio is nearly constant at low pulse frequencies (roughly 20) while the ratio appears to be directly proportional to power at higher pulse frequencies.

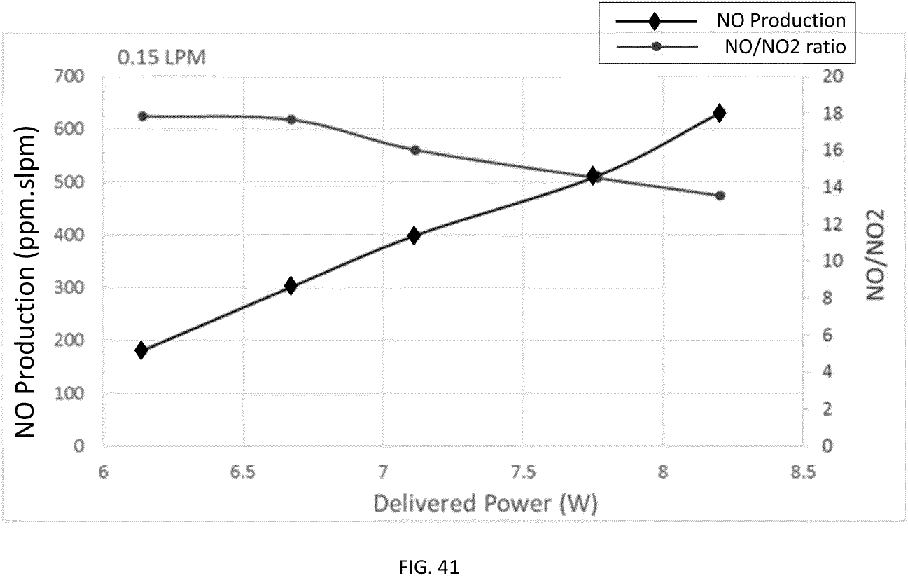

FIG. 41 depicts a graph showing the performance of an exemplary system with applied power on the X axis, NO production on the left vertical axis and NO/NO₂ ratio on the right vertical axis. All data in FIG. 41 were collected with a reactant gas flow rate of 150 ml/min. The relationship between applied power and NO production is linearly proportional. As power and NO production increase, the NO/NO₂ ratio decreases.

The ignition time for a microwave plasma ball is roughly 0.5 µsec, whereas the ignition time for an electrical discharge is 15-40 µsec. Rapid initiation time of a microwave plasma ball can allow for, first, the shorter ignition time enables shorter plasma pulses at higher frequencies to be utilized. Second, variance in plasma initiation time is much shorter, resulting in less variance in NO production from pulse to pulse. Another benefit is that any ozone that would be formed during plasma initiation would be less with a short plasma initiation time. This would manifest as improved NO/NO2 ratio and less NO2 produced.

### Clinical Applications

There are also a variety of clinical applications of inhaled NO, for example, in an ambulatory setting, including the following:
WHO Group 1 PAH - Potential to subtype e.g., idiopathic, familial etc., pediatric PAH, and PAH during pregnancy (avoids toxicity from PAH drugs)
WHO group 2 PAH - Selected well-controlled patients with left heart failure (risk of pulmonary edema, and LVAD recipients with right heart disease (RHD) and pulmonary hypertension (PH) (Orphan)
WHO group 3 PH - PH-ILD or subtype ILD e.g., IPF, CT-related ILD, cHP, etc., PH-COPD, and Combined pulmonary fibrosis emphysema (CPFE)
WHO group 4 Chronic Thromboembolic PH (CTEPH) - Improve right heart disease (RHD)
Sarcoidosis
Right heart dysfunction, diverse etiology - Afterload reduction even in absence of pulmonary hypertension (PH), and Etiologies include ischemic heart disease, valvular disease etc.
Bacterial, fungal, and viral infections
Infectious diseases, such as cystic fibrosis e.g. pseudomonas, B. Cepacia, NTM, Multiple Drug-resistant tuberculosis, Non-tuberculous mycobacterial infection (NTM), and Bronchiectasis
Bridge to lung and/or heart transplant - Addresses pulmonary hypertension (PH), oxygenation, RVD etc
Post lung and/or heart transplant - Reduces pulmonary vascular resistance and contributes to the prevention of bacterial infections
High altitude medicine - To address mountain sickness, High altitude pulmonary edema (HAPE), and reduce hypoxic pulmonary vasoconstriction
Military field applications, such as inhalation injury, cardiopulmonary resuscitation/shock, and High-altitude sickness including during flight
Cardiopulmonary Resuscitation - reverses acute PH due to pulmonary vasoconstriction increasing cardiac output (compressions)
With stored blood or hemoglobin oxygen carriers to prevent complications
During cardiopulmonary bypass to prevent complications
With ECMO to reduce the use of heparin and kidney protection

It will be appreciated that several of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

What is claimed is:

1. A system for generation of nitric oxide (NO), comprising:
a microwave generator configured to produce microwave energy of varying pulse duration, pulse frequency, and power level;
a microwave cavity configured to utilize the microwave energy to generate a plasma ball within a flow of reactant gas containing nitrogen and oxygen flowing through the microwave cavity to produce a product gas containing NO;
at least one stub positioned in the microwave cavity and configured to focus the microwave energy at a location at which the plasma ball is formed; and
a controller in electrical communication with the microwave generator, the controller being configured to control the microwave generator to initiate and maintain the plasma ball such that the plasma ball is suspended in the flow of reactant gas and does not contact a surface of the at least one stub and the microwave cavity.

2. The system of claim 1, wherein the controller is configured to control a concentration of NO in the product gas using one or more control parameters to adjust at least one of the pulse duration, the pulse frequency, and the power level of the microwave energy and a reactant gas flow rate, the control parameters being related to at least one of the reactant gas, the product gas, an inspiratory gas into which at least a portion of the product gas flows, a prescribed amount of NO, and a patient receiving at least the portion of the product gas.

3. The system of claim 1, further comprising a reaction chamber positioned within the microwave cavity such that the plasma ball is positioned within the reaction chamber.

4. The system of claim 3, wherein the reaction chamber is configured to have an independent volume of gas therein.

5. The system of claim 3, wherein the reaction chamber is configured to have an independent volume of gas therein that is less than a volume of gas within the microwave cavity.

6. The system of claim 5, wherein the volume of gas in the reaction chamber allows for a decrease in transit time and NO2 formation due to the volume of gas in the reaction chamber being less than the volume of gas in the microwave cavity.

7. The system of claim 3, further comprising a vacuum chamber associated with the reaction chamber to initiate the plasma ball below atmospheric pressure.

8. The system of claim 1, further comprising a valve upstream of the microwave cavity and a pump downstream of the microwave cavity, the valve and the pump working in combination to decrease a pressure in the microwave cavity.

9. The system of claim 8, wherein the controller is configured to control one or more of the valve and the pump to control the pressure in the microwave cavity.

10. The system of claim 1, wherein the reactant gas includes NO to facilitate plasma formation.

11. The system of claim 1, further including a cooling component configured to cool the reactant gas to increase NO production.

12. The system of claim 11, wherein a temperature of the reactant gas is reduced up to 50° C.

13. The system of claim 1, wherein the microwave generator includes a first antenna configured to initiate a plasma and a second antenna configured to sustain the plasma.

14. A system for generating nitric oxide (NO), comprising:
a microwave generator configured to produce microwave energy of varying pulse duration, pulse frequency, and power levels;
a microwave cavity configured to utilize the microwave energy to generate a plasma ball within a flow of reactant gas containing nitrogen and oxygen flowing through the microwave cavity to produce a product gas containing NO;
a reaction chamber positioned within the microwave cavity and having a gas volume less than a gas volume of the microwave cavity;
at least one stub positioned in the microwave cavity and configured to focus the microwave energy at a location inside the reaction chamber such that a plasma ball is formed therein; and
a controller in electrical communication with the microwave generator, the controller being configured to control the microwave generator to initiate and maintain the plasma ball in the reaction chamber.

15. The system of claim 14, wherein the plasma ball is suspended in the flow of reactant gas and does not contact a surface of the at least one stub and the microwave cavity.

16. The system of claim 14, wherein the controller is configured to control a concentration of NO in the product gas using one or more control parameters to adjust at least one of the pulse duration, the pulse frequency, and the power level of the microwave energy, a reactant gas flow rate, and microwave cavity pressure, the control parameters being related to at least one of the reactant gas, the product gas, an inspiratory gas into which at least a portion of the product gas flows, and a patient receiving at least the portion of the product gas.

17. A method of generating nitric oxide (NO), comprising:
generating a plasma ball from a flow of reactant gas through a microwave cavity using microwave energy directed therein for producing a product gas containing nitric oxide from the flow of the reactant gas through the microwave cavity;
focusing the microwave energy to a focal point in the microwave cavity using at least one stub positioned in the microwave cavity such that focal point is the location of the plasma ball; and
controlling, using a controller, an amount of nitric oxide in the product gas using one or more parameters as input to a control algorithm used the controller to control the generation of the plasma ball,
wherein the plasma ball is suspended in the flow of reactant gas and does not contact a surface of the at least one stub and the microwave cavity.

18. The method of claim 17, wherein focusing the microwave energy further comprises focusing the microwave energy in a reaction chamber positioned within the microwave cavity such that the plasma ball is positioned within the reaction chamber.

19. The method of claim 17, wherein the controller is configured to control a concentration of NO in the product gas using one or more control parameters to adjust at least one of a pulse duration of the microwave energy, a power level of the microwave energy, a reactant gas flow rate, and reaction chamber pressure, the control parameters being related to at least one of the reactant gas, the product gas, an inspiratory gas into which at least a portion of the product gas flows, and a patient receiving at least the portion of the product gas.

20. The method of claim 17, further comprising cooling the reactant gas using a cooling component to increase NO production.

21. The method of claim 20, wherein a temperature of the reactant gas is reduced up to 50° C.
